(19) United States
(12) Patent Application Publication
(10) Pub. No.: US 2011/0021990 A1
(43) Pub. Date: Jan. 27, 2011

(54) MICROPUMP AND METHOD FOR MANUFACTURING THEREOF

(76) Inventors: Thierry Navarro, Gland (CH); Florent Junod, Veigy Foncenex (FR)

Correspondence Address: JOHN ALEXANDER GALBREATH, 2516 CHESTNUT WOODS CT, REISTERSTOWN, MD, 21136, US

(21) Appl. No.: 12/572,300

(22) Filed: Oct. 2, 2009

(30) Foreign Application Priority Data
Jul. 23, 2009 (IB) PCT/IB2009/006336
Sep. 29, 2009 (IB) PCT/IB2009/006996

(51) Int. Cl. A61M 5/142; A61M 1/00; F04B 17/00; B23P 15/00

(52) U.S. Cl. 604/151; 417/437; 417/321; 29/888.02

(57) ABSTRACT

A micropump comprises a valve system having one gasket (10) shaped to define three cavities (12, 12a, 12b) connected respectively to a piston chamber, an inlet port and an outlet port of the pump. A valve switching element (16) having at least one groove (17) is movably mounted on the gasket such that, during piston instrokes, said groove moves along a part of the gasket adjacent to the cavities connected respectively to the piston chamber and the inlet port of the pump, thereby creating a leakage between said cavities so that fluid is sucked into the piston chamber during a piston instroke. During piston outstrokes, said groove moves along a part of the gasket adjacent to the cavities connected respectively to the piston chamber and the outlet port of the pump, thereby creating a leakage between said cavities so that fluid is expelled out of the piston chamber through the outlet port during a piston outstroke.

### TECHNICAL FIELD

The present invention concerns a micropump and a method for manufacturing thereof. This pump is intended to be used in any industrial, chemical, pharmaceutical or medical application such as enteral, parenteral, IV pumps and is particularly adapted to be used as an insulin pump given that its internal mechanism is designed for obtaining an ultra small and very light pump while being capable to deliver a very small bolus directly from a loadable penfill cartridge.

### BACKGROUND OF THE INVENTION

Insulin pumps are widely known in the prior art and are an alternative to multiple daily injections of insulin by an insulin syringe or an insulin pen. Insulin pumps make it possible to deliver more precise amounts of insulin than can be injected using a syringe. This supports tighter control over blood sugar and Hemoglobin A1c levels, reducing the chance of long-term complications associated with diabetes. This is predicted to result in a long term cost savings relative to multiple daily injections.

Some insulin pumps comprise internal receiving means for an insulin cylindrical penfill cartridge. US2007/0167912 describes a pump of this kind comprising a plunger engagement device mounted inside the pump to face a plunger of an insulin penfill cartridge when said cartridge is inserted into the receiving means of the pump. The plunger engagement device is configured to attach to the cartridge plunger when urged together. This device is connected to a flexible piston rod arranged to push the cartridge plunger inside the penfill cartridge along a preset distance so that an insulin dose can be expelled out of the cartridge. A major drawback of this pump lies on the complexity of the driving mechanism that actuates the piston rod. This mechanism is made of numerous components whose arrangement inside the pump makes it difficult to minimize its size. As an insulin pump needs to be worn most of the time, pump users may find it uncomfortable or unwieldy. Besides, assembling all the parts of the pump as described therein is a time-consuming process which further requires strenuous quality control as numerous interacting parts increase the risk of failure making the pump less reliable.

Another disadvantage of this kind of pump occurs when the piston pushes directly the cartridge plunger inside the penfill cartridge along its longitudinal axis, the plunger tending to move irregularly along said axis as an important, irregular and uncontrolled friction exists. This phenomenon is better known as the so-called “stick slip” effect and has a direct impact on the pump accuracy.

These disadvantages have been solved, to a large extend, by a volumetric pump mechanism as described in WO2006056828. This volumetric pump comprises a first and a second piston which are mounted inside a first and a second hollow cylindrical part (chamber) to be movable along the longitudinal axis of said cylindrical parts, while being synchronized to each other such that a specific amount of fluid is sucked in during the instroke of the first piston, while the same amount of fluid is expelled during the outstroke of the second piston. The first and the second hollow cylindrical part are assembled end-to-end facing each other to form a housing. A valve disc (valve system), which comprises an inlet and outlet port connected respectively to an inlet and outlet T-shaped channel, is mounted between the first and second piston inside the housing and is arranged to be animated by a combined bidirectional linear and angular movement which couples the pistons strokes with the movement of the valve system. More precisely, the linear movement of the disc produces a to-and-fro sliding of the cylindrical housing along the axis of the pistons causing an alternate instroke of the first and second pistons followed by an alternate outstroke of the first and second pistons inside their respective chambers while its angular movement synchronizes the first piston chamber filling phase with the second piston releasing phase. This synchronization is achieved by an inlet and outlet T-shaped channel located inside the valve disc which connects alternately the inlet port to the first and second chamber, and the first and second chamber to the outlet port when said channels overlap alternately an inlet aperture and an outlet aperture located across the diameter of both cylindrical parts adjacent to the lateral sides of the disc. The flow of the fluid released by this pump is quasi-continuous.

A major drawback of this volumetric pump is that the inlet and outlet aperture, arranged to be aligned alternately with the inlet and outlet T-shaped channel, are located across the diameter of both cylindrical parts adjacent to the lateral sides of the valves disc. As a result, the volume reduction of the first and second chamber is limited to the size of the apertures below which it would be insufficient to guarantee a normal flow delivery.

Another drawback of this pump stems from the fact that the inlet and the outlet channels are mounted on the valve disc to which a linear and angular movement is imparted. As a result, the inlet and outlet ports and the tubes connected thereto are continuously moving under working condition which may be troublesome for pump users who may find it uncomfortable to wear.

### SUMMARY OF THE INVENTION

An aim of the present invention is to simplify the internal mechanism of the pump in order to reduce its dimensions, to improve its reliability as well as its accuracy.

This aim is achieved by a micropump comprising a pump housing containing at least one piston chamber, at least one piston arranged to be linearly actuable to move back and forth inside the chamber, the micropump having at least one inlet port and at least one outlet port arranged so that a fluid can be sucked through the inlet port into the chamber during an instroke of the piston and expelled from the chamber through the outlet port during an outstroke of the piston. The pump further includes a valve system that comprises, on the one hand, at least one gasket that is shaped to define at least three cavities connected respectively to the piston chamber, the inlet port and the outlet port of the pump, and on the other hand, a valve switching element mounted on the gasket to allow relative movement between the gasket and the valve switching element. At least one groove or other recess is arranged on the valve switching element such that, during piston instrokes, said groove or recess moves along or across a part of the gasket that is adjacent to the cavities connected respectively to the piston chamber and the inlet port of the pump, thereby creating a first communication allowing leakage between said cavities so that fluid is sucked into the piston chamber during a piston instroke. During piston outstrokes, said groove or recess moves along or across a part of the gasket that is adjacent to the cavities connected respectively to the piston chamber and the outlet port of the pump, thereby creating a second communication allowing leakage between said cavities so that fluid is expelled out of the piston chamber through the outlet port during a piston outstroke.

Another aim of the present invention is to provide a method for manufacturing the pump comprising a minimum number of steps so as to reduce its production costs and to improve its reliability.

This aim is achieved by an injection moulding process which comprises the following steps: (a) injecting a mouldable plastic material capable of forming a substantially rigid element into a mould cavity assembly for obtaining a base part of the pump housing; (b) placing a seal mould matrix on said base part of the pump housing where the valve system is to be mounted, said mould matrix being designed to reproduce the shape of the gasket of the pump; and (c) injecting into said matrix a mouldable rubber-elastic material in a flowable state, the rubber-elastic material polymerizing in the mould matrix while being bound to the pump housing to form said gasket.

### BRIEF DESCRIPTION OF THE DRAWINGS

The invention will be better understood thanks to the following detailed description of several embodiments with reference to the attached drawings, in which:

FIG. 1 shows a see-through perspective view of a micropump comprising one piston housing according to a first embodiment of the invention;

FIG. 2 shows a see-through perspective bottom view of the pump of FIG. 1;

FIG. 3 shows a bottom view of the pump of FIG. 1;

FIG. 4 shows an exploded view of a valve switching device composed of a disc and a gasket;

FIG. 5 shows an elevational view of the pump of FIG. 1 connected to a driving mechanism and a cartridge;

FIG. 6 shows a top view of FIG. 5;

FIG. 7 shows a cross-sectional view of the pump taken on the line A-A in FIG. 5;

FIG. 8 shows a cross-sectional view of the pump taken on the line D-D in FIG. 6;

FIG. 9 shows a cross-sectional view of the pump taken on the line B-B in FIG. 5;

FIG. 10 shows a cross-sectional view of the pump taken on the line C-C in FIG. 5;

FIG. 11 shows a perspective view of the pump driving mechanism;

FIG. 12 shows an exploded view of the pump case unit, its cartridge and a removable lid securely holding on its bottom part the pump;

FIG. 13 shows a perspective view of a patch pump incorporating the pump and its driving mechanism according to the first embodiment of the invention;

FIG. 14 shows a perspective view of a system adapted to connect the pump to a cannula;

FIG. 15 shows the disposable part of the patch pump of FIG. 13;

FIG. 16 shows a perspective view of the pump without the disposable part;

FIG. 17 shows a perspective view of a patch pump according to a variant;

FIG. 18 shows a perspective view of an automatic device for inserting the cannula into the patient body;

FIG. 18a shows a partial cross-sectional view of FIG. 18;

FIG. 19 shows the automatic device of FIG. 18 mounted on the patch pump of FIG. 13;

FIG. 20a shows a front view of the upper part of the pump connected to its driving mechanism and insulin cartridge just before the beginning of a pumping cycle when there is no pumping movement;

FIG. 20a′ shows a cross-sectional view taken respectively on the lines A-A, B-B and C-C in FIG. 20a;

FIG. 20b shows a similar view of FIG. 20a during a piston instroke of the pump;

FIG. 20b′ shows cross-sectional views taken respectively on the lines A-A, B-B and C-C in FIG. 20b;

FIG. 20c shows a similar view of FIG. 20a at the end of the piston instroke of the pump;

FIG. 20c′ shows cross-sectional views taken respectively on the lines A-A, B-B and C-C in FIG. 20c;

FIG. 20d shows a similar view of FIG. 20a during a piston outstroke of the pump;

FIG. 20d′ shows cross-sectional views taken respectively on the lines A-A, B-B and C-C in FIG. 20d;

FIG. 21 shows an exploded view of a micropump and its driving mechanism according to a second embodiment of the invention;

FIG. 22 shows a perspective view of the pump when connected to its driving mechanism;

FIG. 23 shows an exploded view of the pump of FIG. 21;

FIG. 24 shows an elevational view of the pump and its driving mechanism;

FIG. 25 shows a cross-sectional view of the pump taken on the line A-A in FIG. 24;

FIG. 26 shows a top view of FIG. 24;

FIG. 27 shows a cross-sectional view of the pump taken on the line B-B in FIG. 26;

FIG. 28 shows a perspective view of a micropump and its driving mechanism according to a variant of the second embodiment of the invention;

FIG. 29 shows a bottom view of FIG. 28;

FIG. 30 shows an elevational view of the pump and its driving mechanism;

FIG. 31 shows a cross-sectional view of the pump taken on the line A-A in FIG. 30;

FIG. 32 shows a top view of FIG. 28;

FIG. 33 shows a cross-sectional view of the pump and its driving mechanism taken on the line B-B of FIG. 32;

FIG. 34 shows an exploded bottom view of the pump;

FIG. 35 shows an exploded top view of the pump;

FIG. 36 shows a see-through perspective view of a micropump comprising a first and a second piston according to a third embodiment of the invention;

FIG. 37 shows a see-through bottom view of FIG. 36;

FIG. 38 shows an exploded view of a valve switching device composed of a disc and seal element according to this third embodiment;

FIG. 39 shows a perspective view of the pump of FIG. 36 connected to its driving mechanism;

FIG. 40 shows a top view of FIG. 39;

FIG. 41 shows a cross-sectional view of the pump taken on the line A-A in FIG. 40;

FIG. 42 shows a cross-sectional view of the pump taken on the line B-B in FIG. 40;

FIG. 43a shows a front view of the upper part of FIG. 39 just before the beginning of a pumping cycle when there is no pumping movement;

FIG. 43a′ shows cross-sectional views of the pump taken respectively on the lines A-A, B-B and C-C in FIG. 43a;

FIG. 43b shows a similar view of FIG. 43a during an instroke of the first piston and an outstroke of the second piston;

FIG. 43b′ shows cross-sectional views of the pump taken respectively on the lines A-A, B-B and C-C in FIG. 43b

FIG. 43c shows a similar view of FIG. 43a at the end of the first piston instroke and the second piston outstroke;

FIG. 43c′ shows cross-sectional views of the pump taken respectively on the lines A-A, B-B and C-C in FIG. 43c

FIG. 43d shows a similar view of FIG. 43a during an outstroke of the first piston and an instroke of the second piston;

FIG. 43d′ shows cross-sectional view of the pump taken respectively on the lines A-A, B-B and C-C in FIG. 43d.

FIG. 44 shows a basic schematic view of a valve switching device for a pump according to another embodiment of the invention;

FIG. 45 shows a basic schematic view of the valve switching device for a pump according to a further embodiment of the invention;

FIG. 46 shows a basic schematic view of the valve switching device for a pump according to a yet further embodiment of the invention;

FIG. 47 shows a see-through perspective view of a pump according to an even further embodiment of the invention;

FIG. 48 shows a see-through perspective view of a cylindrical valve holder inside a pump housing of the pump of FIG. 47;

FIG. 49 shows a perspective view of the cylindrical valve holder comprising gaskets;

FIG. 50 shows a see-through perspective view of the pump housing inside which is axially mounted a piston;

FIG. 51 shows an axially cross-sectional view of FIG. 50;

FIGS. 52, 53 and 54 show see-through perspective views of a pump according to a variant of FIGS. 47 to 51.

### DETAILED DESCRIPTION OF EMBODIMENTS OF THE INVENTION

### First Embodiment of the Invention

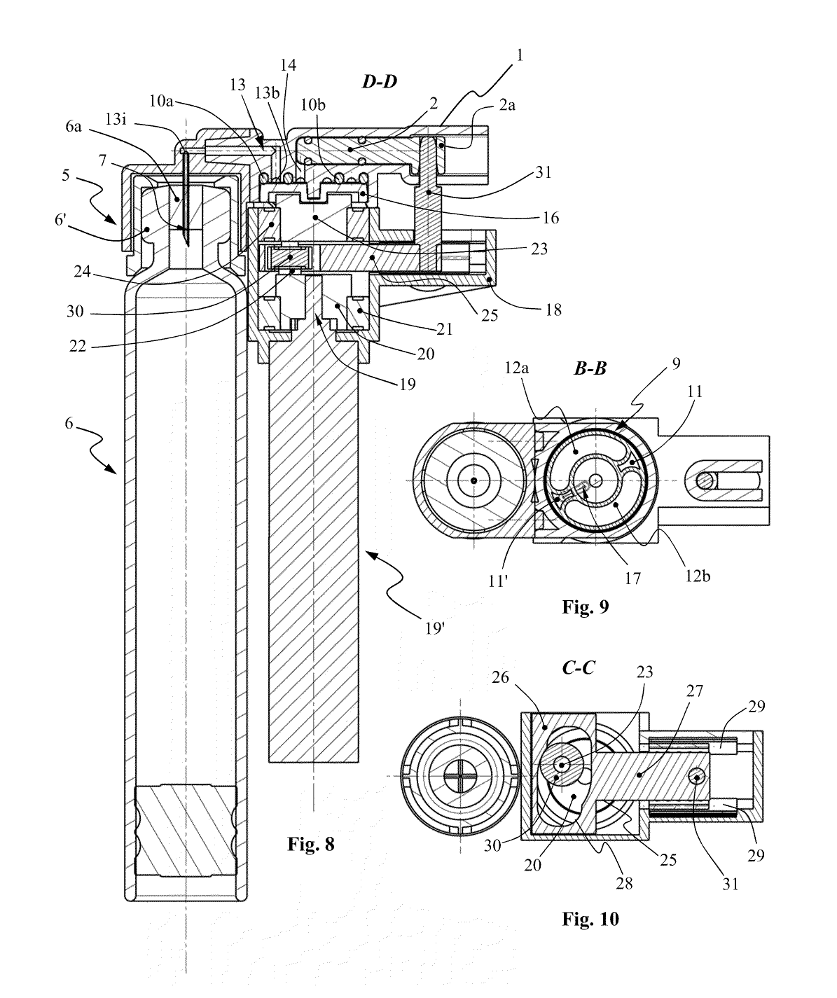

According to the first embodiment of the present invention as shown in FIGS. 1 to 13, the micropump comprises a preferably disposable plastic molded housing 1 having a piston chamber 1′ (FIG. 7) within which a pump piston 2 is mounted so as to be movable back and forth inside chamber 1′, and a cylindrical cap 5 for receiving the head 6′ of a penfill cartridge 6 (FIG. 8). A needle 7 is axially mounted inside the cylindrical cap 5 and is adapted to pierce the cartridge head 6′ when the latter is urged into said cap 5. For this purpose, an inner part 6a of the cartridge head 6′ is made of a soft material to ease the introduction of needle 7 into the cartridge content.

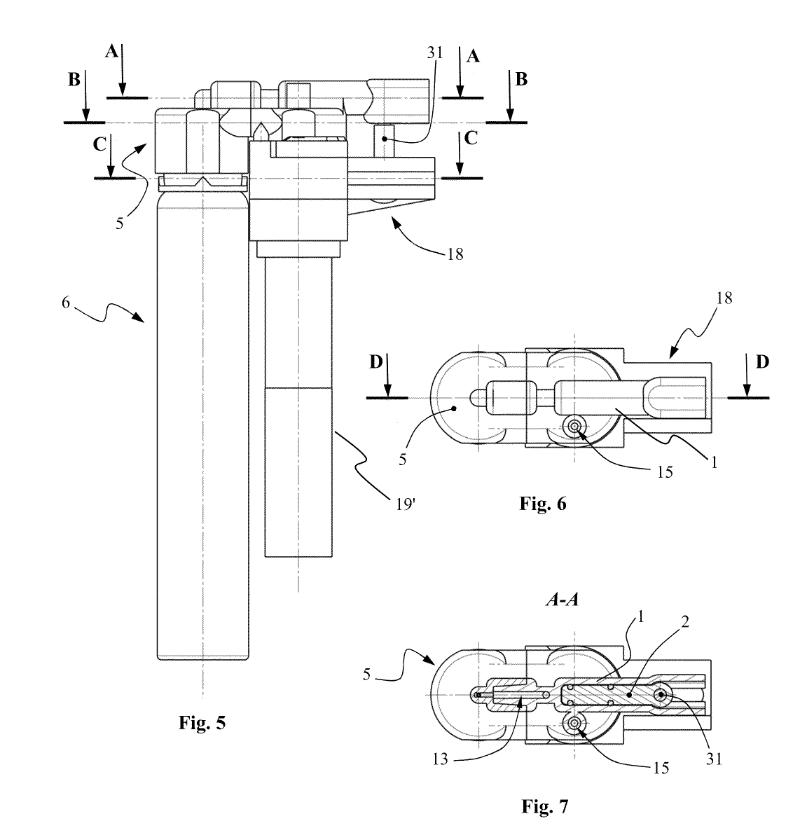

The bottom part of the micropump comprises a cylindrical recess 9 (FIG. 9) adapted to receive a valves system to open and close in turn an inlet and an outlet port 13i, 15o of the pump during a pumping cycle. For this purpose, recess 9 has a bottom surface adapted to receive a gasket 10 (FIG. 4) that comprises two concentric rings, namely an inner and an outer ring 10a, 10b attached together by a first and a diametrically opposed second sealing part 11, 11′. The gasket 10 is preferably obtainable by an injection moulding process.

A rotary disc 16, that comprises a rectilinear groove 17, is rotatably mounted against the gasket 10. Said gasket 10 is shaped as to obtain arcuate inlet and outlet cavities 12a, 12b symmetrically opposed and curved with respect to the rotation axis of the rotary disc 16, and a circular cavity 12 axially centred on said axis. Cavities 12a, 12b are defined by the inner ring 10a, the outer ring 10b and the two sealing parts 11, 11′ of the gasket 10, while the circular cavity 12 is defined by the gasket inner ring 10a.

Referring now to FIGS. 3 and 8, the housing 1 of the micropump further comprises an L-shaped inlet channel 13 which has an inner extremity 13i, which can be seen as the inlet port 13i of the pump, and that is connected to the needle 7, and an outer extremity 13b located on the bottom surface of the cylindrical recess 9 inside the arcuate inlet cavity 12a. Another channel 14 is arranged to extend parallel to one part of channel 13 from piston chamber 1′ to the bottom surface of recess 9 inside central cavity 12. The pump also comprises an outlet channel 15 which has an inner end 15a located on the bottom surface of recess 9 inside the arcuate outlet cavity 12b. The outer end of the outlet channel 15 corresponds to the outlet port 15o of the pump.

Rectilinear groove 17 of the disc 16 is arranged to extend radially to both sides of the gasket inner ring 10a such that, during a piston instroke, the piston chamber 1′ is connected to the inlet port 13i of the pump, as rotation of the disc 16 allows the rectilinear groove 17 to move along and to extend across a part of the inner ring 10a that is adjacent to the central cavity 12 and the arcuate inlet cavity 12a creating a first communication allowing leakage between said cavities 12, 12a. During a piston outstroke, the piston chamber 1′ is connected to the outlet port 15o of the pump, as the disc 16 further rotates to allow the rectilinear groove 17 to move along and to extend across a part of the inner ring 10a that is adjacent to the central cavity 12 and the arcuate outlet cavity 12b creating a second communication allowing leakage between said cavities 12, 12b. Thus, the valves system is actuated as a function of the angular movement of disc 16.

Referring to FIGS. 8 to 10, the pump driving mechanism comprises a supporting structure 18 having a lower part adapted to receive a rotary shaft 19 of a motor 19′. A first rotatable element 20 is coaxially mounted on the shaft 19 and is laterally secured by a first ball bearing assembly 21. The lower part of a second shaft 22 is mounted eccentrically on the rotatable element 20 and extends vertically therefrom through a substantially rectangular-shaped aperture 28 of a T-shaped sliding tray 25 to have its upper part connected eccentrically to a second rotatable element 23 that is axially aligned with the first rotatable element 20 and laterally secured by a second ball bearing assembly 24. The T-shaped sliding tray 25 is arranged on the supporting structure 18 to be actuable by a to-and-fro linear movement in a direction perpendicular to the rotating axis of the rotary shaft 19.

For this purpose, as shown in FIG. 10, the T-shaped sliding tray 25 comprises a first rectangular part 26 extending perpendicular to a second rectangular part 27. The first part 26 comprises the substantially rectangular-shaped aperture 28 while both lateral sides of second part 27 rest on two rails 29 secured to the supporting structure 18 by any suitable means. A ball bearing assembly 30 is fitted around the eccentric shaft 22 inside the aperture 28 to impart the to-and-fro linear movement to the sliding tray 25 when the eccentric shaft 22 rotates. A driving pin 31 is arranged to protrude vertically from the second rectangular part 27 of the sliding tray 25 through the piston head 2a (FIG. 8) in order to actuate the to-and-fro linear movement of the piston 2 inside its chamber 1′.

The aperture 28 of the sliding tray 25 is shaped as to have a specific contour such that the sliding tray 25 is actuated when the ball bearing 30 moves along the contour of aperture 28 to produce a controlled pumping cycle over the valve switching cycle.

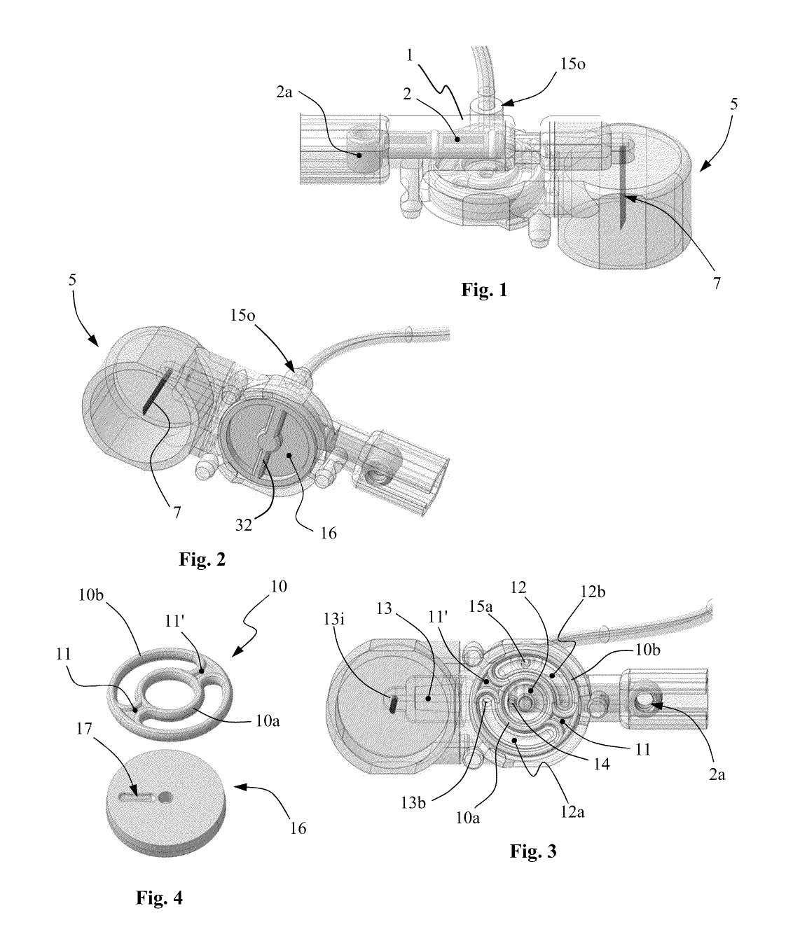

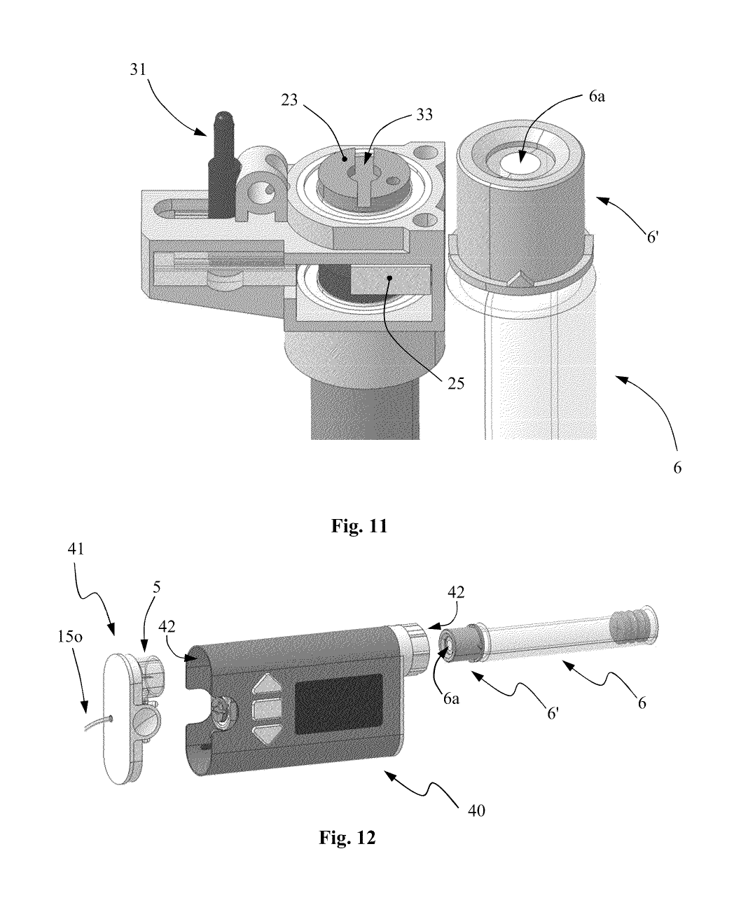

With reference to FIG. 2, the disc 16 comprises on its bottom a rectilinear bulge 32 extending along the entire diameter of disc 16 and having a round-shaped part centred on the disc rotation axis. This bulge 32 is adapted to be tightly secured in a corresponding groove 33 located on the upper part of the second rotatable element 23 of the pump driving mechanism (FIG. 11). The disc 16, which comprises the rectilinear groove 17, is thus continuously rotating at controlled speed through an angle of 360° during a pumping cycle. This rectilinear groove 17, which extends radially to both sides of the gasket inner ring 10a, is therefore arranged to move along the entire circumference of said inner ring 10a during a pumping cycle (FIG. 9), thereby creating a communication allowing leakage between the piston chamber 1′, and in turn the inlet and outlet ports 13i, 15o of the pump.

As shown in FIG. 12, the micropump of this first embodiment and its driving mechanism are mounted inside a case unit 40. This case unit 40 comprises a disposable removable lid 41 securely holding on its bottom part the micro pump as described above and illustrated particularly in FIG. 3. The driving mechanism of the pump is mounted inside the case unit 40 so that its piston driving pin 31 and its rotatable disc 16 are aligned to be inserted respectively through the piston head 2a and into the cylindrical recess 9 of the pump when lid 41 is securely fitted on case unit 40. The latter further comprises a cylindrical holding cartridge unit 42 into which penfill cartridge 6 is insertable. Said holding cartridge unit 42 is arranged to receive at one end the cylindrical cap 5 of the pump. The soft inner part 6a of the cartridge head 6′ is thus pierced by the needle 7 axially mounted inside the cap 5, when penfill cartridge 6 is inserted inside the holding unit 52 and its head 6′ is urged into said cap 5.

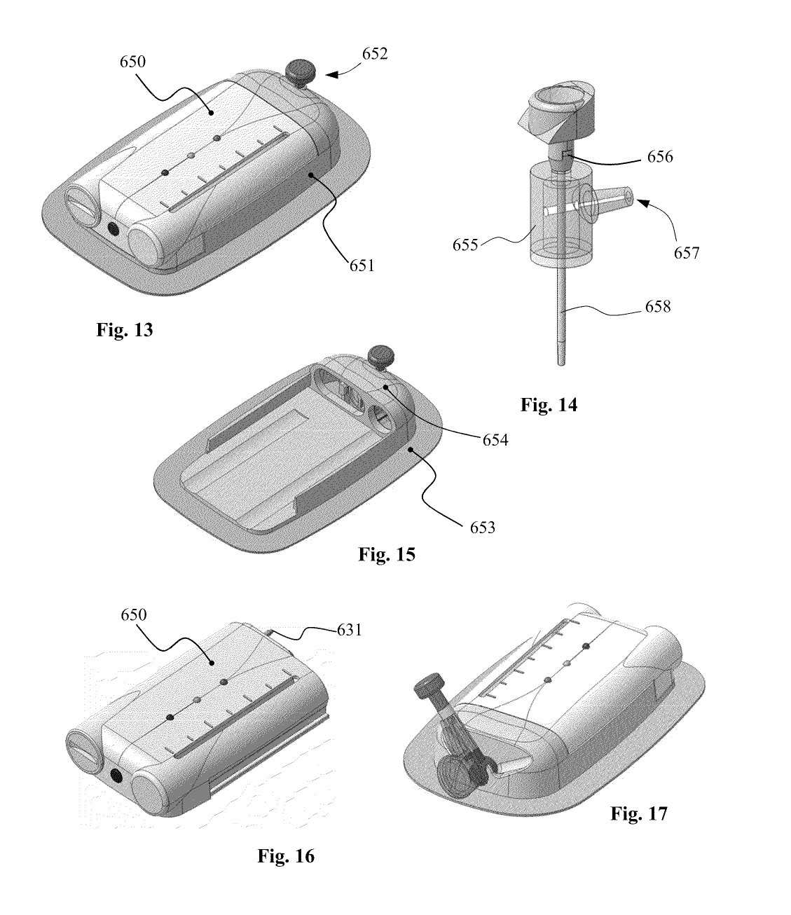

As shown in FIGS. 13 to 17, the pump can be incorporated in a wearable patch pump. In this configuration, a reusable driving unit 650 incorporates the driving mechanism of the pump, a battery and electronic components while a disposable receiving unit 651 comprises a case pump 654 incorporating the disposable pump of this first embodiment, and an adhesive membrane 653 adapted to be stuck on a part of a patient body. FIG. 14 shows a piece 655 that is adapted to be mounted inside the case pump 654 to connect a tube 657 to the outlet port 15o of the pump (FIG. 19). A cannula needle 658 is axially and slidably mounted inside this piece 655 and its upper part is connected to a locking device 656. The cannula needle 658 is inserted into the skin when a knob 652 connected to the upper end of said cannula 658 is pressed, whereupon the locking device 656 is clipped to a corresponding part (not shown) located inside the case pump 680 to hold in place the cannula 658 while the needle is withdrawn by pulling the knob 652. FIG. 17 shows a variant of the patch pump which comprises a system which allows controlling the depth of the insertion of the cannula 658 into the skin by adapting its angle of insertion.

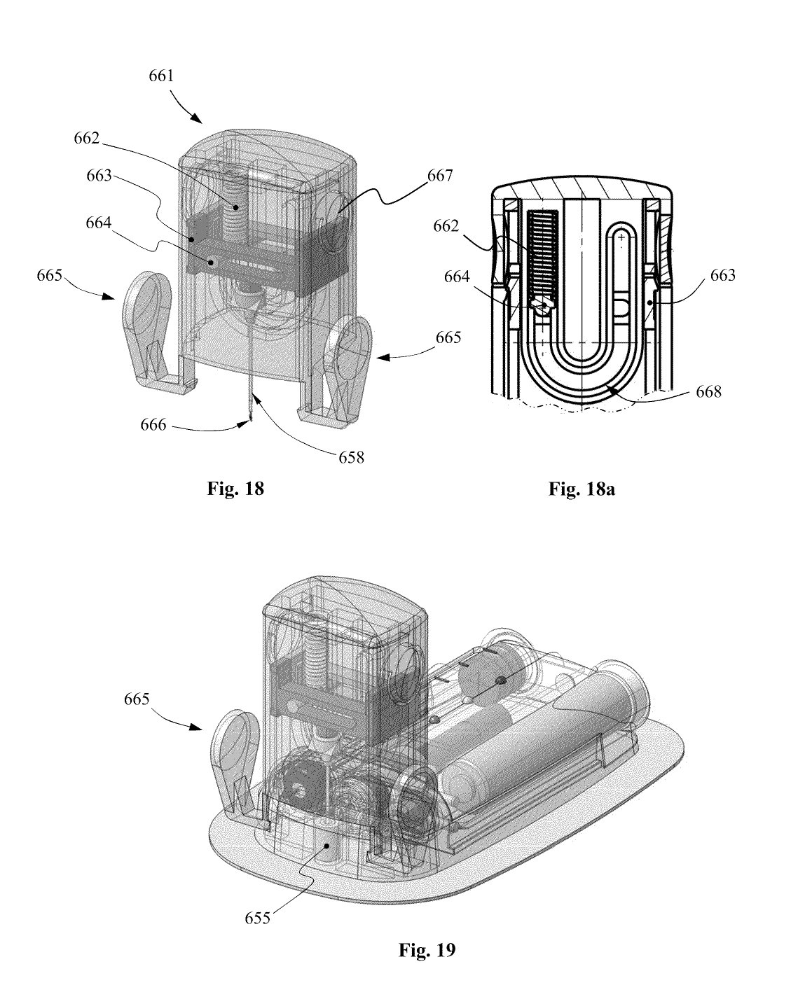

FIG. 18 shows an automatic device for inserting the cannula into the skin. This device comprises a housing 661 inside which is slidably mounted a tray 663 actuable along a vertical axis by a spring 662 arranged to expand inside a U-shaped part 668. The cannula needle 658 is realisably connected to the bottom of the tray 663. One end of the spring 662 is connected to a rod 664 that is arranged across the tray 663 to move along two longitudinal apertures 669 performed on both longitudinal sides of tray 663 as the spring 662 expands along the entire U-shaped part 668. Two push buttons 667 are located on both lateral sides of the automatic device to release the tray 663 when actuated. The automatic device can easily be fitted and secured on the case pump 654 by applying a small pressure. The two push buttons 667 are then pressed together, thereby releasing the tray 663 which is actuated downwards along with the cannula 658 by the expansion of the spring 662 to the point the rod 664 reaches the bottom of the U-shaped part, whereupon the cannula has been inserted into the skin at the desired depth and the locking device 656 is clipped to a corresponding part (not shown) located inside the case pump 680 to hold in place the cannula 658. At this stage, the spring 662 further expands inside the U-shaped part and pushes the tray 663 upwards, thereby withdrawing the needle 666 from the cannula 658. The automatic device is then removed from the patch pump by simply pressing simultaneously two releasing means 666 arranged on both lateral sides of said device.

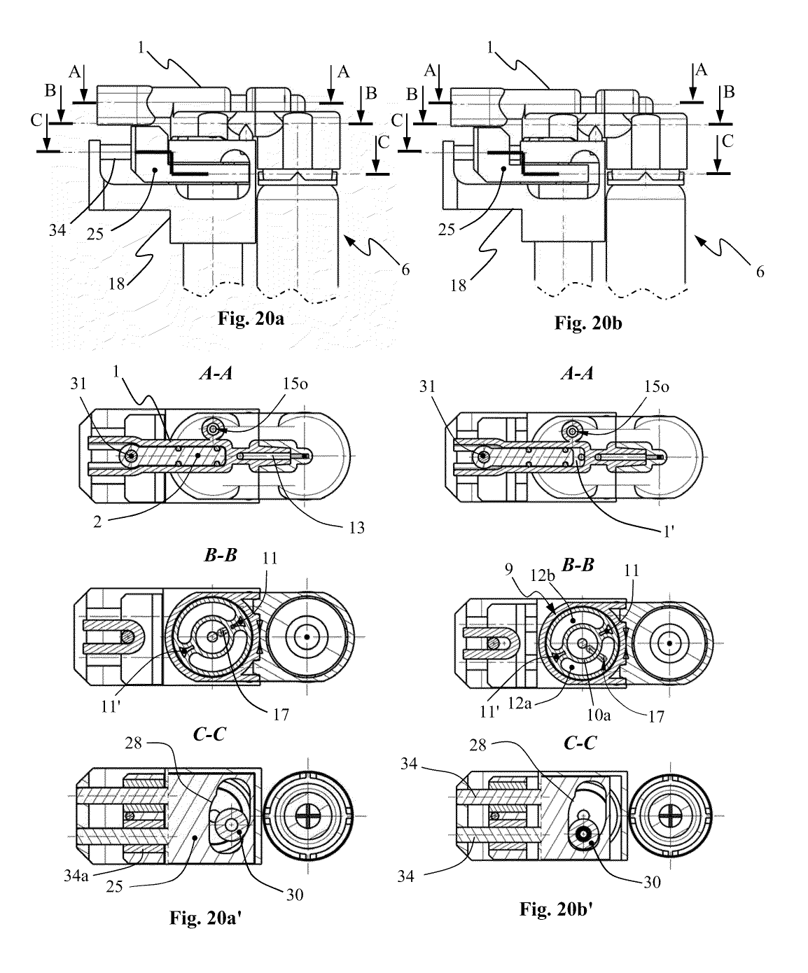

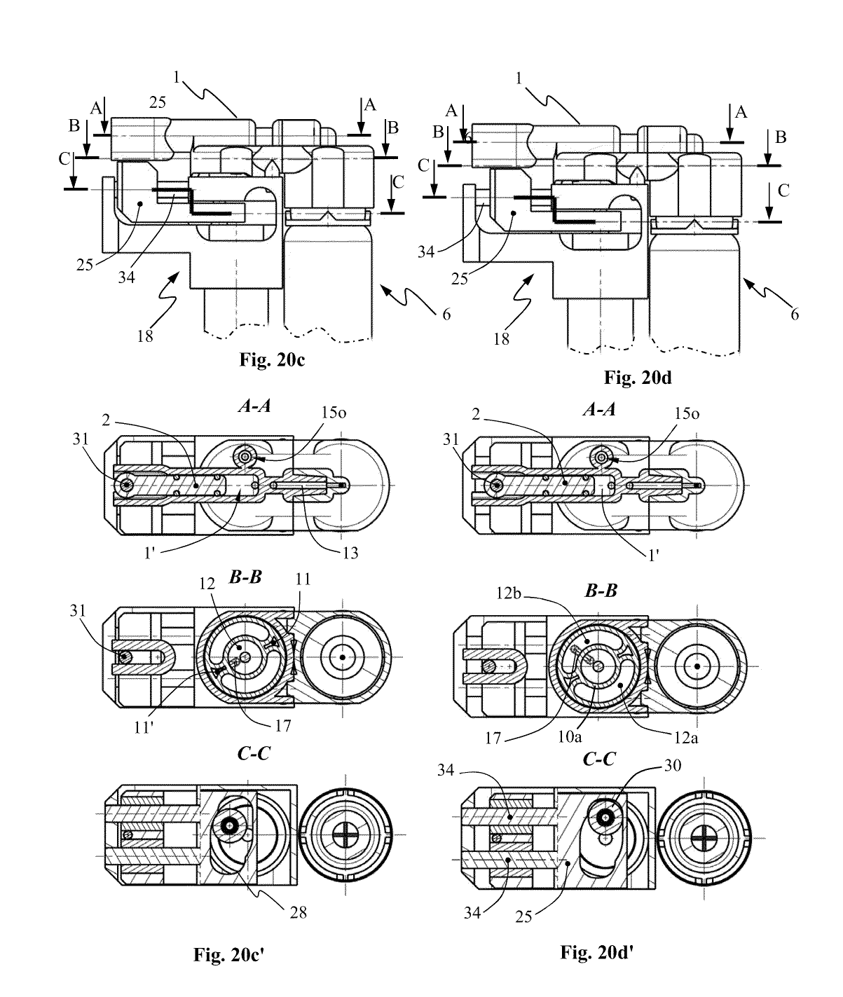

Detailed description of the pump according to this first embodiment as it goes through the principal phases of a pumping cycle will now be described particularly with reference to FIGS. 20a to 20d. In these Figures, the pump driving mechanism slightly differ from the pump driving mechanism illustrated by FIGS. 8 to 10 by the shape of the supporting structure 18 and the sliding tray 25 as well as by the sliding means which consist of two rods 34 protruding parallel to each other and perpendicular to one lateral side of the supporting structure 18, these rods 34 being slidably adjusted inside two corresponding borings 34a performed on one side of sliding tray 25.

FIG. 20a and its corresponding cross-sectional views (FIG. 20a′) show the pump and its driving mechanism just before the beginning of a pumping cycle when there is substantially no movement of the piston 2 and when the valve switching occurs. At this stage of the pumping cycle, the sliding tray 25 has been pushed by the ball bearing 30 to one of its farthest lateral positions (cross-sectional view C-C of FIG. 20a) and the rectilinear groove 17 of the disc 16 is angularly positioned to extend radially under the first sealing part 11 of the gasket 10 (cross-sectional view B-B of FIG. 20a). In this configuration, the piston 2 is entirely sealed from the inlet and outlet ports 13i, 15o of the pump.

Valve switching is performed by the rotation of the disc 16 which brings its rectilinear groove 17 from one side to the other side of the first sealing part 11 of gasket 10, whereupon the rectilinear groove 17 creates a communication allowing leakage between arcuate inlet cavity 12a and central cavity 12 in order to connect the piston chamber 1′ to the L-shaped channel 13 of the pump.

From this instant, the ball-bearing 30 is in contact with the border of aperture 28 (cross-sectional view C-C of FIG. 20b) and pushes backwards the tray 25 which causes simultaneously an instroke of the piston 2 by means of the piston driving pin 31 (cross-sectional view A-A of FIG. 20b), while the rectilinear groove 17 of disc 16 extends radially to both sides of the inner ring 10a and moves along a part thereof adjacent to both arcuate inlet cavity 12a and central cavity 12 when disc 16 rotates through an angle of approximately 150° (cross-sectional view B-B of FIG. 20b). During this rotation, L-shaped inlet channel 13 and channel 8 of the pump as shown in FIG. 8 are permanently connected to each other. As a result, a medicinal fluid, such as insulin, contained in the penfill cartridge 6 is sucked through the needle 7, passing in turn through L-shaped channel 13, rectilinear groove 17 and channel 8 to fill the piston chamber 1′.

FIG. 20c and its corresponding cross-sectional views show the pump and its driving mechanism at the end of the piston instroke when there is substantially no movement of the piston 2 and the valve switching occurs. At this stage of the pumping cycle, the sliding tray 25 has been pushed by the ball bearing 30 to the other of its farthest lateral positions (cross-sectional view C-C of FIG. 20c) and the rectilinear groove 17 of disc 16 is angularly positioned to extend radially under the second sealing part 11′ of gasket 10 (cross-sectional view B-B of FIG. 20c). In this configuration, the piston 2 is entirely sealed from the inlet and outlet ports 13i, 15o of the pump.

Valve switching is performed by rotating the disc 16 to bring its rectilinear groove 17 from one side to the other side of the second sealing parts 11′ of gasket 10, whereupon the rectilinear groove 17 creates a communication allowing leakage between arcuate outlet cavity 12b and central cavity 12 in order to connect the piston chamber 1′ to the outlet port 15o of the pump.

From this instant, the ball-bearing 30 is in contact with the border of aperture 28 and pushes forwards the tray 25 which causes an outstroke of the piston 2 by means of the piston driving pin 31 (cross-sectional view A-A of FIG. 20d), while the rectilinear groove 17 of disc 16 extends radially to both sides of the inner ring 10a and moves along a part thereof adjacent to both arcuate outlet cavity 12b and central cavity 12 when disc 16 further rotates through an angle of approximately 150° (cross-sectional view B-B of FIG. 20d). During this rotation, outlet channel 15 is permanently connected to outlet port 15o of the pump. As a result, the medicinal fluid is expelled from the piston chamber 1′ passing in turn trough channel 8, rectilinear groove 17, arcuate outlet cavity 12b and outlet channel 15. At this point, another pumping cycle begins as described above. The pump as designed in this first embodiment delivers an intermittent flow of a medicinal substance.

### Second Embodiment of the Invention

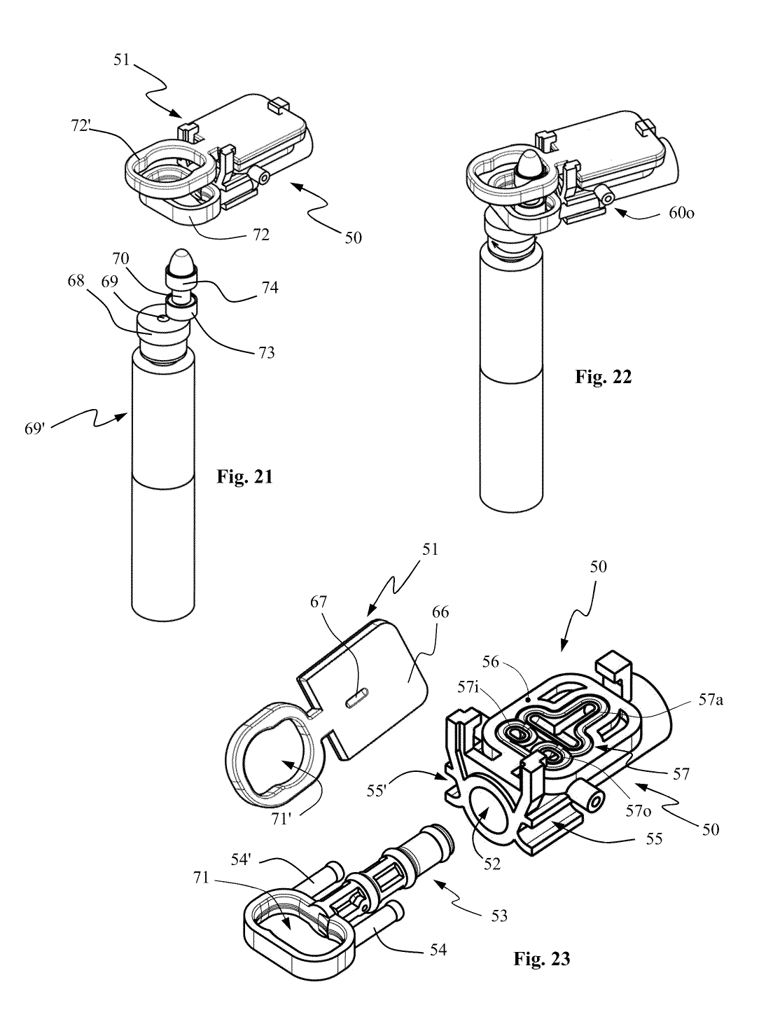

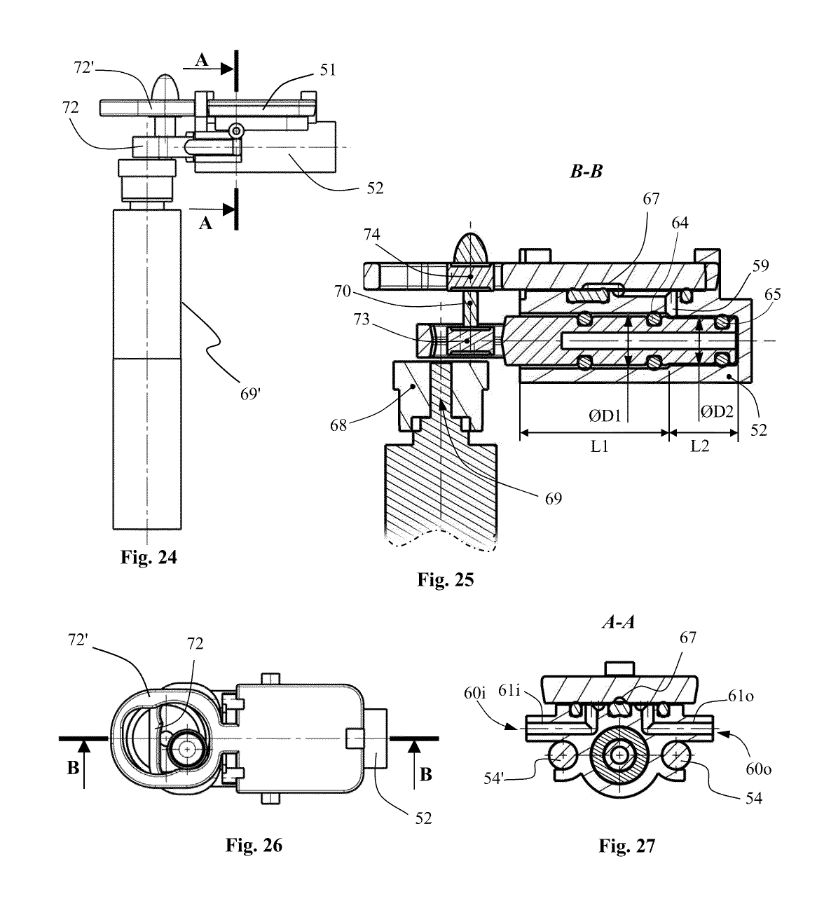

FIGS. 21 to 27 show a micropump and its driving mechanism according to a second embodiment of the invention. This pump is advantageously designed to dispense with the guiding elements of its driving mechanism as described in the first embodiment of the invention particularly in order to minimize the size of the pump and to simplify its manufacturing process.

For this purpose, this pump is made of a lower part 50 and an upper part 51. As shown in FIG. 23, the lower part 50 of the pump contains a hollow cylindrical housing 52 (piston chamber) inside which is arranged a piston 53. Two cylindrical protruding parts 54, 54′ arranged on both lateral sides of the piston 53 are slidably mounted along two half-cylindrical guidance means 55, 55′ located on both lateral sides of the lower part 50 of the pump so that piston 53 can be actuable by a to-and-fro linear movement in a single plane. Lower part 50 of the pump has an upper surface 56 adapted to receive a gasket 57. The gasket 57 is shaped as to define annular-rectangular-shaped (or O-shaped) inlet and outlet cavities 57i, 57o that are connected to an inlet and an outlet port 60i, 60o of the pump by an inlet and an outlet channel 611, 610, the gasket 57 further defining a generally T-shaped cavity 57a connected to the piston chamber by a channel 59 (FIG. 25). The inlet and outlet cavities 57i, 57o are arranged next to each other along their common longitudinal axis that is oriented in a direction perpendicular to the piston movement, while the chamber cavity 57a is arranged to have a rectilinear part thereof adjacent to one lateral side of both the inlet and the outlet cavity 57i, 57o.

Referring to FIG. 25, the piston chamber 52 of the pump has a first and a second axial extension L1, L2 having two different diameters D1, D2. A first and a second O-ring 64, 65 are arranged around the piston 53 to move respectively along the first and the second axial extension L1, L2, during a pumping cycle. The piston chamber volume is therefore given by “((D1−D2)/2)²×π×L” where L is the length of the piston stroke, and is therefore much smaller than the piston chamber volume given by the entire diameter of the piston chamber of the pump as described in the first embodiment of the invention. A smaller bolus can therefore be delivered increasing the pump accuracy.

Referring again to FIG. 23, the upper part 51 of the pump comprises a flat bottom surface 66 that is mounted to rest on the upper surface 56 of lower part 50 of the pump and to be actuable by a to-and-fro linear movement in a direction perpendicular to the piston movement. A rectilinear groove 67 is arranged on its flat bottom surface 66 so that one part of the groove 67 extends partially above the T-shaped cavity 57a, while the other part of said groove 67 partially extends in turn above the 0-shaped inlet and outlet cavities 57i, 57o, as it moves back and forth perpendicularly along the longitudinal axis of said 0-shaped inlet and outlet cavities 57i, 57o.

FIG. 21 shows a driving mechanism adapted to impart to-and-fro linear movements to the piston 53 and the upper part 51 of the pump. This driving mechanism comprises a rotatable element 68 mounted around the rotary shaft 69 of a motor 69′. A second shaft 70 is eccentrically mounted on the rotatable element 68 to extend vertically therefrom and is adapted to protrude through two substantially rectangular-shaped apertures 71, 71′ of two superposed guiding elements 72, 72′ that form a part of respective piston 53 and upper part 51 of the pump. The two longitudinal axes of the two rectangular-shaped apertures 71, 71′ are perpendicular to each other so that the guiding element 72 actuates a full piston instroke or oustroke, whereupon guiding element 72′ actuates the upper part 51 in a direction perpendicular to the piston movement.

For that purpose, a first ball bearing assembly 73 is fitted around the second shaft 70 in order to rest against a part of the contour of aperture 71 of the piston guiding element 72, while a second ball bearing assembly 74 is fitted around said shaft 70 in order to rest against a part of the contour of aperture 71′ of the upper part guiding element 72′. Rotation of eccentric shaft 72 imparts to-and-fro linear movement to piston 53 as ball bearing 73 moves along the entire contour of aperture 71 of the piston guiding element 72, and a perpendicular to-and-fro linear movement to the upper part 51 of the pump, as the ball bearing 74 moves along the entire contour of aperture 71′ of the upper part guiding element 72′.

The piston chamber is connected to the inlet port 60i of the pump as the rectilinear groove 67 of the upper part 51 moves along a part of the gasket 57 that is adjacent to both the inlet cavity 57i and the T-shaped cavity 57a during a piston instroke, thereby creating a first communication allowing leakage between said cavities 57i, 57a so that fluid is sucked from inlet port 60i passing in turn through inlet channel 61i, the inlet cavity 57i, the rectilinear groove 67, the T-shaped cavity 57a and the channel 59 to fill the piston chamber. During a piston outstroke, the piston chamber is connected to the outlet port 60o of the pump, as the rectilinear groove 67 of the upper part 51 moves further along a part of the gasket 57 that is adjacent to both the outlet cavity 57o and the T-shaped cavity 57a, thereby creating a second communication allowing leakage between said cavities 57o, 57a so that the fluid is expelled from the piston chamber, passing in turn through the channel 59, the T-shaped cavity 57a, the rectilinear groove 67, the outlet cavity 57o and the outlet channel 610 out of the outlet port 60o.

The lower part 50 of the pump can be obtainable by an injection moulding process which comprises the following steps: (a) injecting a mouldable plastic material capable of forming a substantially rigid element into a mould cavity assembly for obtaining the base of lower part 50; (b) placing a seal mould matrix on the upper part of the base of lower part 50 where the valve system is to be mounted, the seal mould matrix being designed to reproduce the shape of the above-mentioned gasket 57; and (c) injecting into said matrix a mouldable rubber-elastic material in a flowable state, the rubber-elastic material polymerizing in the mould matrix while being bound to the upper part of the base of lower part 50.

The gasket 57 can also be obtainable by a separate injecting moulding process and added on a corresponding groove arranged on the upper surface 56 of lower part 50.

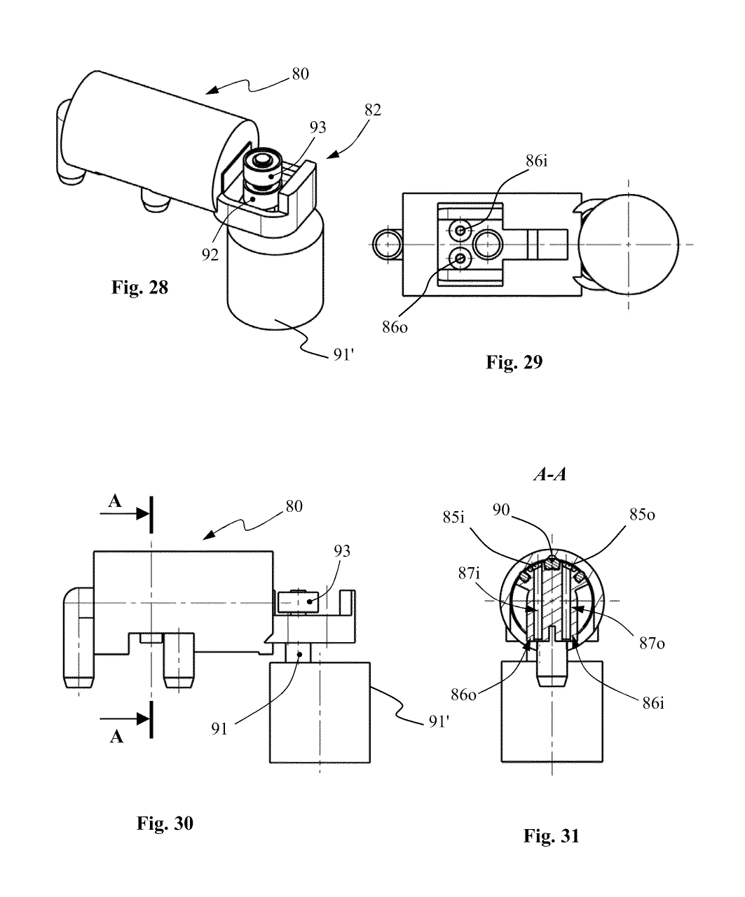

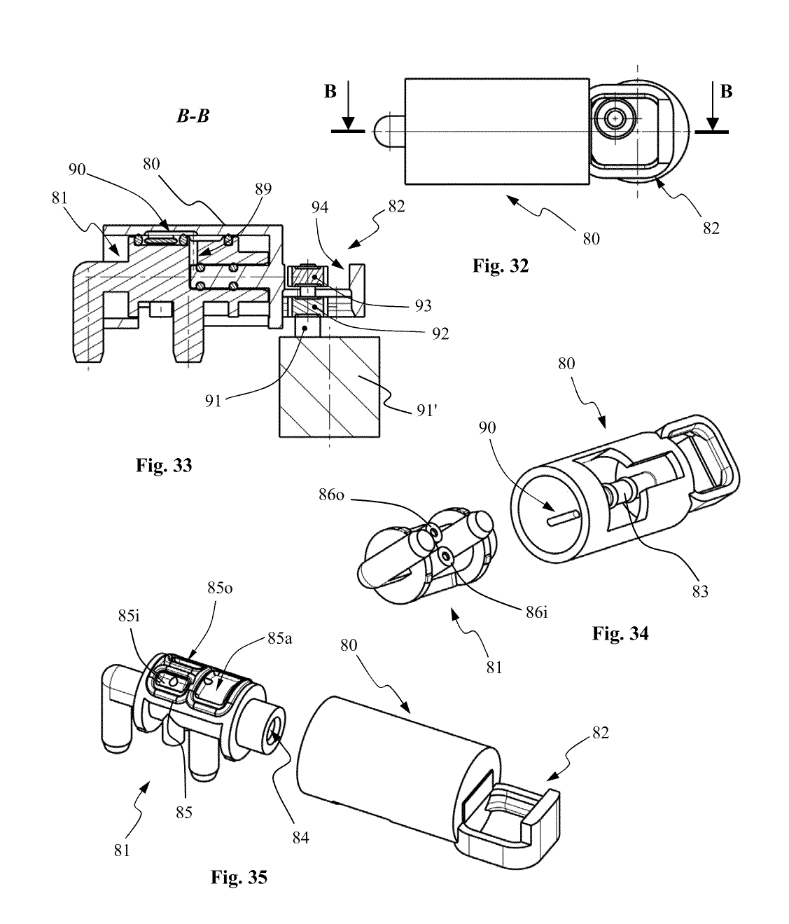

FIGS. 28 to 35 show a micropump according to a variant of the second embodiment of the invention. This pump comprises a hollow cylindrical housing 80 that is adapted to receive a valve holder 81 (FIGS. 34 and 35) and to be actuable by to-and-fro linear and to-and-fro angular movements. A piston 83 is axially mounted within the hollow cylindrical housing 80 to project inside a corresponding hollow cylindrical chamber 84 of the valve holder 81. As shown by FIG. 35, a gasket 85 is arranged on the outer surface of a cylindrical part of the valve holder 81 and is configured to define O-shaped inlet and outlet cavities 85i, 85o and a rectangular chamber cavity 85a. Inlet and outlet cavities 85i, 85o are aligned adjacent to each other and to the chamber cavity 85a. Inlet and outlet cavities 85i, 85o are connected to the inlet and outlet ports 86i, 86o of the pump by respective inlet and outlet channels 87i, 87o (FIG. 31), while the chamber cavity 85a is connected to the piston chamber by a channel 89 (FIG. 33). A rectilinear groove 90 is arranged on the inner surface of the pump housing 80 (FIG. 34) such that one part of said groove 90 extends partially above the chamber cavity 85a, while the other part of groove 90 partially extends alternately above the inlet and outlet cavities 85i, 85o, as the pump housing 80 rotates back and forth about its rotating axis.

To-and-fro linear and angular movements of the pump housing 80 are imparted by a driving mechanism that comprises a shaft 91 mounted eccentrically on a motor 91′ and around which a first and a second ball bearing 92, 93 are fitted (FIG. 33). This eccentric shaft 91 is arranged to extend through a substantially square aperture of an O-shaped guiding element 82 connected to the pump housing 80. This guiding element 82 is mounted to be axially unbalanced with the piston axis such that during a pumping cycle the first ball bearing 92 swings the pump housing 80 around its rotating axis, while the second ball bearing 93 imparts a to-and-fro linear movement to the piston 83 inside its chamber 84.

Different sequences of the pump and its driving mechanism of FIGS. 28 to 33, as they go through a pumping cycle will now be described in more detail. The first ball bearing 92 of the eccentric shaft 91 moves along a first part of the inner contour of the guiding element 82 as the shaft 91 rotates to 90 degrees (FIG. 28), thereby rotating the pump housing 80 such that its rectilinear groove 90 extends across a part of the gasket 85 that is adjacent to the inlet cavity 85i and the chamber cavity 85a of the pump creating a first communication allowing leakage between said cavities 85i, 85a. The second ball bearing 93 is then brought into contact with a projecting part 94 of the guiding element 82 as the eccentric shaft 91 further rotates. A piston instroke is then actuated by means of the second ball bearing 93 which pushes against the guiding element projecting part 94, so that fluid can be sucked from the inlet port 86i of the pump, passing in turn through an inlet channel 87i (FIG. 31), the inlet cavity 85i, the rectilinear groove 90, the chamber cavity 85a and the channel 89 to fill the piston chamber. As the piston 83 reaches the end of its instroke, the first ball bearing 92 moves along a second part of the inner contour of the guiding element 82 which is diametrically opposed to the first part, thereby rotating the pump housing 80 in an opposite direction such that its rectilinear groove 90 extends across a part of the gasket 85 that is adjacent to the outlet cavity 85o and the chamber cavity 85a of the pump creating a second communication allowing leakage between said cavities 85o, 85a. The second ball bearing 93 is then brought into contact with the lateral side of the pump housing 80 as the eccentric shaft 91 further rotates. A piston outstroke is then actuated by means of the second ball bearing 93 which pushes against the lateral side of the pump housing 80 so that fluid can be released from the piston chamber, passing in turn through the channel 89, the chamber cavity 85a, the rectilinear groove 90, the outlet cavity 85o, and the outlet channel 87o to be expelled out of the outlet port 86o of the pump.

According to another variant, the above described second embodiment and its variant can be adapted to comprise a second piston chamber. For this purpose, a second pump identical to the first pump of the second embodiment or identical to its variant is coupled to its corresponding first pump and is arranged symmetrically with respect to a median plane. In this configuration, first and second pistons and the valve system are guided by one or two common guiding element such as described in the second embodiment or its variant such that a specific amount of fluid is sucked into the first piston chamber during first piston instrokes, while the same amount of fluid is expelled out of the second piston chamber during second piston outstrokes.

### Third Embodiment of the Invention

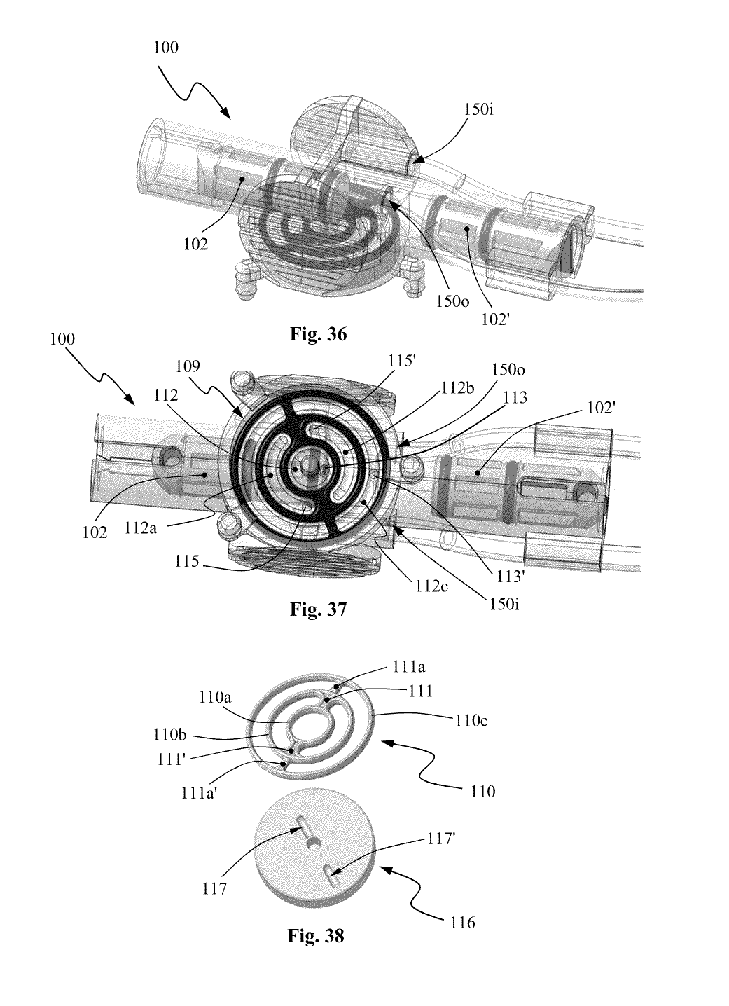

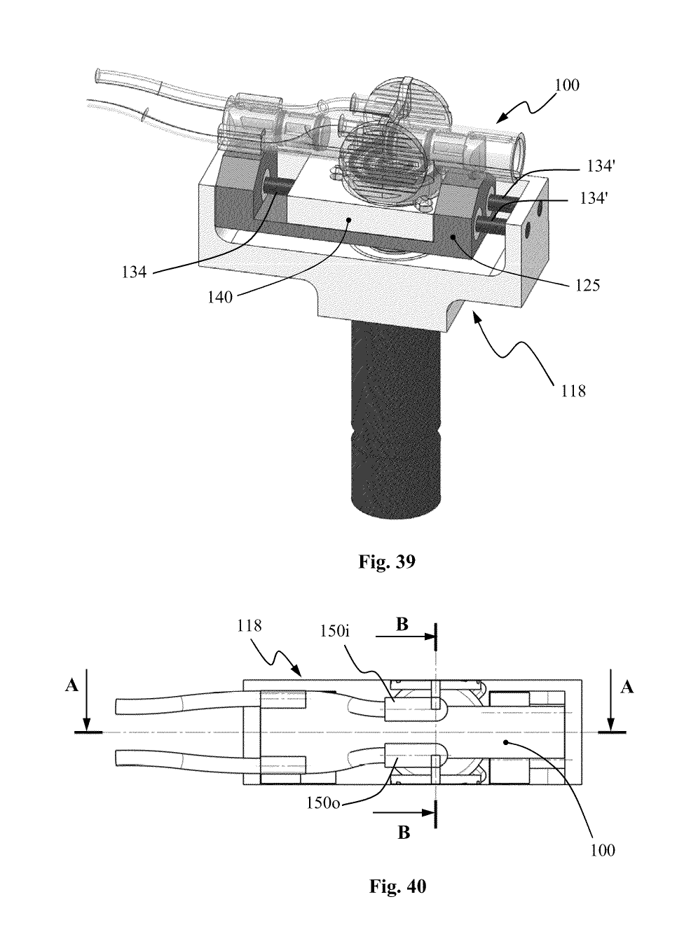

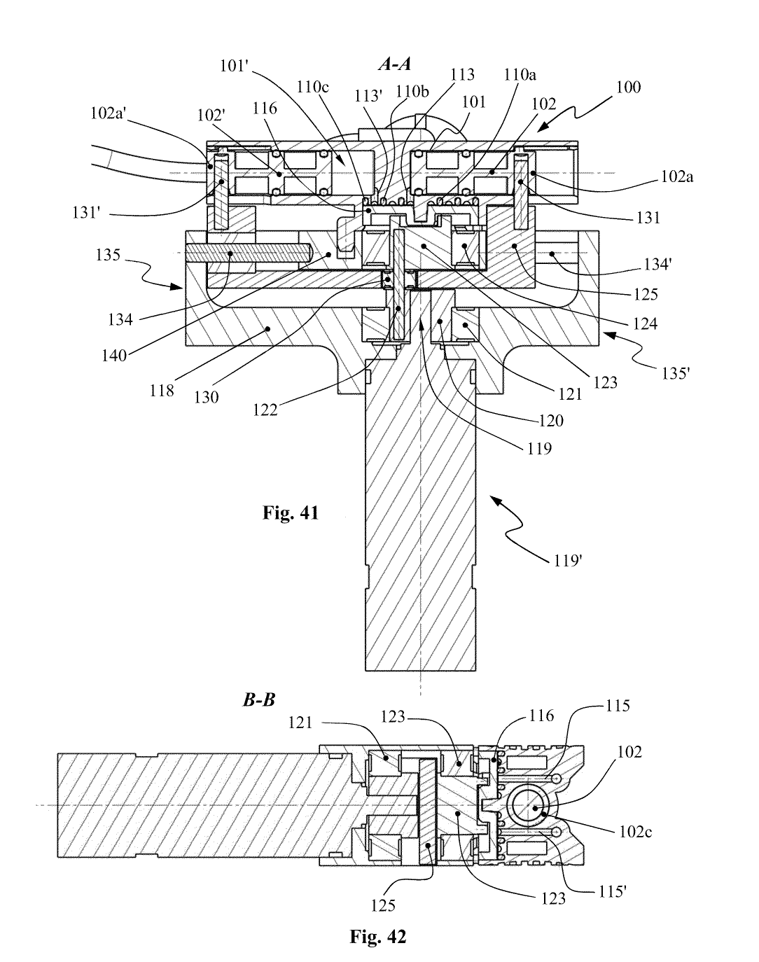

According to a third embodiment of the invention as shown in FIGS. 36 to 43, the micropump is designed for delivering a quasi-continuous flow of a medicinal fluid. This pump comprises a preferably disposable plastic moulded housing 100 having a cylindrical part containing a first and a second chamber 101, 101′ arranged opposite to each other along the longitudinal axis of said cylindrical part (FIGS. 36 and 41). A first and a second piston 102, 102′ are mounted so as to be movable back and forth inside said first and second piston chamber 101, 101′.

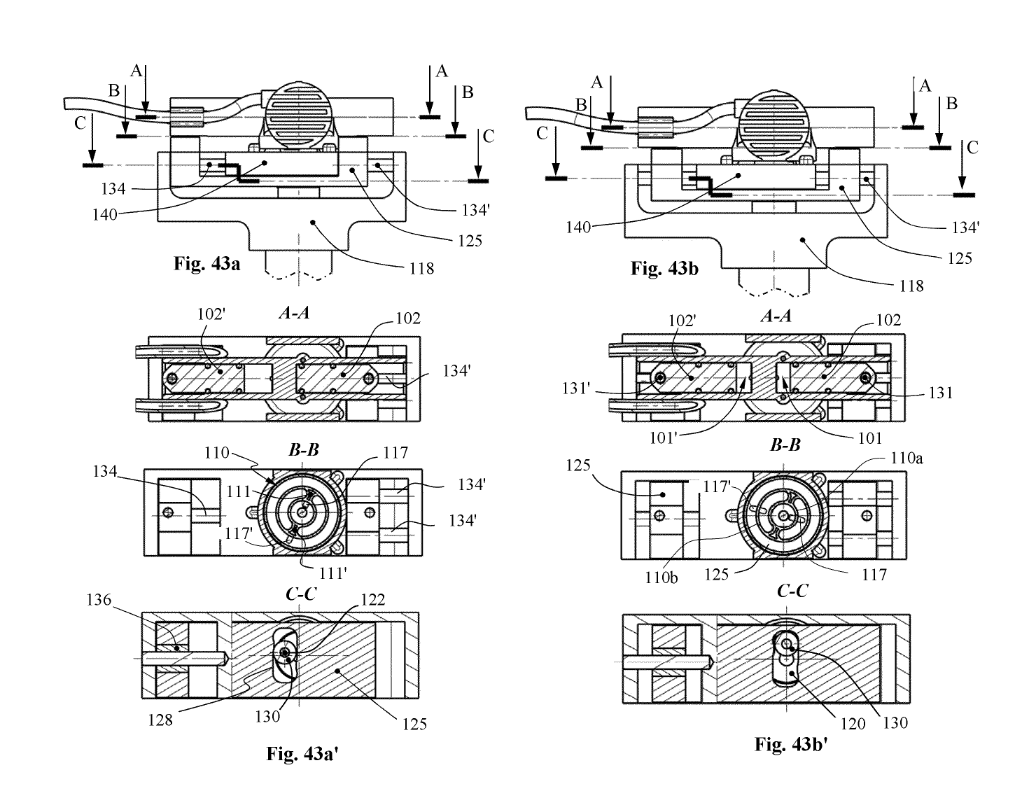

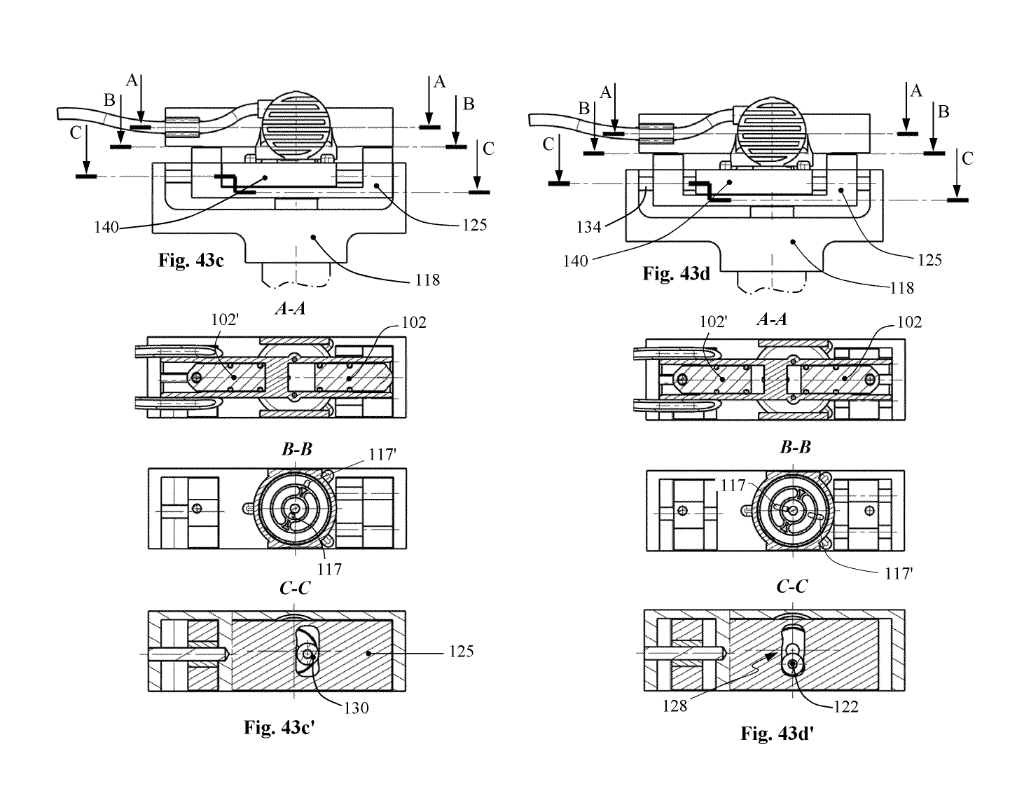

The bottom part of the pump housing 100 comprises a cylindrical recess 109 having a flat bottom surface adapted to fixedly receive a gasket 110 (FIG. 37). The gasket 110 comprises three concentric rings, namely an inner, a middle and an outer ring 110a, 110b, 110c, the inner and middle ring 110a, 110b being connected together by a first and a second sealing part 111, 111′ that are diametrically opposed (FIG. 38). The gasket 110 further comprises two attaching means 111a, 111a′ to hold the outer ring 110c with the middle ring 110b. A central circular cavity 112 is defined by the inner ring 110a, arcuate inlet and outlet cavities 112a, 112b are defined by the inner ring 110a, the middle ring 110b and the two sealing parts 111, 111′ of gasket 110 and a ring-shaped cavity 112c (i.e. cross-sectional view B-B of FIG. 43a) is defined by the gasket middle ring and outer ring 110b, 110c.

As shown in FIG. 41, a first pump channel 113 extends vertically from the piston chamber 101 to the flat bottom surface of recess 109 inside the central circular cavity 112 (FIG. 37). A second pump channel 113′ is arranged to extend vertically from the second piston chamber 101′ to the flat bottom surface of recess 109 (FIG. 41) inside the ring-shaped cavity 112c (FIG. 37). Besides, as shown in FIG. 42, one end of an inlet and an outlet channel 115, 115′ is arranged respectively inside the first arcuate inlet and outlet cavities 112a, 112b, while the other end of said channels 115, 115′ are respectively connected to an inlet and an outlet port 150i, 150o.

With reference to FIG. 38, the gasket 110 is adapted to rest on the surface of a rotatable disc 116. Said disc 116 comprises a first and a second identical rectilinear groove 117, 117′ extending along two segments of its diameter. The first rectilinear groove 117 stands near the rotation axis of the disc 116 and is arranged to extend radially to both sides of the gasket inner ring 110a when disc 116 is rotatably mounted thereon, while the second rectilinear groove 117′ stands at the periphery of disc 116 and is arranged to extend radially to both sides of the gasket middle ring 110b.

With reference to FIG. 41, the mechanism for driving the pump according to this embodiment comprises a U-shaped supporting structure 118 having a lower part adapted to receive a rotary shaft 119 of a stepper motor 119′. A first rotatable element 120 is coaxially mounted on said shaft 119 and is laterally secured by a first ball bearing assembly 121. The lower part of a second shaft 122 is fixedly mounted eccentrically on the rotatable element 120 and extends vertically therefrom through a rectangular-shaped aperture 128 of a U-shaped sliding tray 125 to have its upper part fixedly mounted eccentrically inside a second rotatable element 123 axially aligned with the first rotatable element 120 and laterally secured by a second ball bearing assembly 124 mounted on a supporting piece 140.

The disc 116 is axially secured on the rotatable element 123 by any suitable means such as the ones described in the first embodiment of the invention. Said disc 116 is thus continuously rotating at controlled speed through an angle of 360° during a pumping cycle. The first and second rectilinear grooves 117, 117′ are therefore arranged to move perpendicularly along the entire circumference of respective gasket inner ring and middle ring 110a, 110b during a pumping cycle.

The U-shaped sliding tray 125 is mounted to be actuable by a to-and-fro linear movement across the U-shaped supporting structure 118. To this end, one rod 134 is arranged to protrude perpendicularly from one lateral side 135 of the supporting structure 118 to extend through a corresponding boring 136 located in one side of the tray 125 to be fixedly secured to one lateral side of the supporting piece 140, while a pair of rods 134′ are mounted parallel to each other and protrude perpendicularly from the other lateral side 135′ of the supporting structure 118 to extend through two corresponding borings located in the other side of the sliding tray 125 to be fixedly secured to another lateral side of the supporting piece 140.

To-and-fro linear movement of the sliding tray 125 is imparted by a ball-bearing assembly 130 which is fitted around the eccentric shaft 122 inside the rectangular-shaped aperture 128 of the tray 125 (i.e. cross-sectional view C-C of FIG. 43a). A first and a second piston driving pin 131, 131′ are arranged to protrude vertically from the sliding tray 125 near each of its lateral sides and extend through the heads 102a, 102a′ of the first and the second piston 102, 102′ in order to impart a to-and-fro linear movement to said pistons 102, 102′ inside their respective chambers 101, 101′.

Detailed description of the pump according to this third embodiment as it goes through the principal phases of a pumping cycle will now be described particularly with reference to FIGS. 43a to 43d.

FIG. 43a and its corresponding cross-sectional views (FIG. 43a′) show the pump and its driving mechanism just before the beginning of a pumping cycle when there is substantially no movement of the first and second pistons 102, 102′ and when valves switching occurs. At this stage of the pumping cycle, the sliding tray 125 has been pushed by the ball bearing assembly 130 to one of its farthest lateral positions (cross-sectional view C-C of FIG. 43a) and each of the two rectilinear grooves 117, 117′ of disc 116 are angularly positioned to extend radially under each of the two sealing parts 111, 111′ of the gasket 110 (cross-sectional view B-B of FIG. 43a). In this configuration, the first and second pistons 102, 102′ are entirely sealed from the inlet and outlet ports 150i, 150o of the pump while disc 116 rotates to bring its first and second rectilinear grooves 117, 117′ from one side to the other side of each of the two sealing parts 111, 111′ of gasket 110, whereupon first rectilinear groove 117 creates a leakage between the central circular cavity 112 and the arcuate inlet cavity 112a, while second rectilinear groove 117′ creates a leakage between the ring-shaped cavity 112c and the arcuate outlet cavity 112b. As a result, first piston chamber 101 is connected to the inlet port 150i of the pump while second piston chamber 101′ is connected to the outlet port 150o of said pump.

From this instant, ball bearing assembly 130, which rotates eccentrically, is in contact with the border of rectangular aperture 128 and pushes forwards sliding tray 125 causing an instroke of the first piston 102 and an outstroke of the second piston 102′ (cross-sectional view A-A of FIG. 43b), by means of the first and the second piston driving pins 131, 131′. During this pumping phase, the disc 116 rotates through an angle of approximately 150° moving the first rectilinear groove 117 of the disc 116 along a part of the gasket inner ring 110a that is adjacent to both arcuate inlet cavity 112a and central circular cavity 112 and moving the second rectilinear groove 117′ along a part of the gasket middle ring 110b that is adjacent to both ring-shaped cavity 112c and arcuate outlet cavity 112b (cross-sectional view B-B of FIG. 43b). As a result, the first pump channel 113 is permanently connected to inlet port 150i, while the second pump channel 113′ is permanently connected to the outlet port 150o. A predefined amount of fluid is therefore sucked from the inlet port 150i of the pump, passing in turn through inlet channel 115, arcuate inlet cavity 112a, first rectilinear groove 117, central cavity 112 and first pump channel 113 to fill the first piston chamber 101 during an instroke of the first piston, while a same amount of fluid is expelled from the second piston chamber 101′, passing in turn through second pump channel 113′, ring-shaped cavity 112c, second rectilinear groove 117′, arcuate outlet cavity 112b, outlet channel 115′ to the outlet port 150o during an outstroke of the second piston 102′.

FIG. 43c and its corresponding cross-sectional views (FIG. 43c′) show the pump and its driving mechanism at the end of the instroke and the outstroke of respective first and second pistons 102, 102′ when the valves switching occur.

At this stage of the pumping cycle, the sliding tray 125 has been pushed by the ball bearing assembly 130 to the other of its farthest lateral positions (cross-sectional view C-C of FIG. 43c) and each of the two rectilinear grooves 117, 117′ of the disc 116 are angularly positioned to extend radially under each of the two sealing parts 111, 111′ of gasket 110 (cross-sectional view B-B of FIG. 43c). In this configuration, the first and second pistons 102, 102′ are entirely sealed from the inlet and outlet ports 150i, 150o of the pump while disc 116 rotates to bring its first and second rectilinear grooves 117, 117′ from one side to the other side of each of the two sealing parts 111, 111′ of gasket 110, whereupon the first rectilinear groove 117 creates a leakage between central circular part 112 and arcuate outlet cavity 112b, while second rectilinear groove 117′ creates a leakage between ring-shaped cavity 112c and arcuate inlet cavity 112a. As a result, first piston chamber 101 is connected to the outlet port 150o of the pump while second piston chamber 101′ is connected to the inlet port 150o of said pump.

From this instant, the ball bearing assembly 130, which rotates eccentrically, is in contact with the border of rectangular-shaped aperture 128 and pushes forwards the sliding tray 125 causing an outstroke of the first piston 102 and an instroke of the second piston 102′ (cross-sectional view A-A of FIG. 43d), by means of first and second piston driving pins 131, 131′. During this pumping phase, the disc 116 further rotates through an angle of approximately 150° moving the first rectilinear groove 117 of said disc 116 along a part of the gasket inner ring 110a that is adjacent to both arcuate outlet cavity 112b and central circular cavity 112, and moving the second rectilinear groove 117′ along a part of the gasket middle ring 110c that is adjacent to both ring-shaped cavity 112c and arcuate outlet cavity 112b (cross-sectional view B-B of FIG. 43d). As a result, first pump channel 113 (FIG. 41) is permanently connected to outlet port 150i while second pump channel 113′ is permanently connected to inlet port 150o. Fluid is therefore expelled from the first piston chamber 101 of the pump, passing in turn through the first pump channel 113, central cavity 112, first rectilinear groove 117, arcuate outlet cavity 112b, outlet channel 115′ to the outlet port 150o of the pump, while fluid is sucked from the inlet port 150i of the pump, passing in turn through inlet channel 115, arcuate inlet cavity 112a, second rectilinear groove 117′, central cavity 112 and second pump channel 113′ to fill the second piston chamber 101′ during an instroke of the second piston 102′.

The pump of this embodiment can therefore deliver a quasi-continuous flow of a fluid.

### Other Embodiments

The micropump can be designed to incorporate a valve system having different configurations.

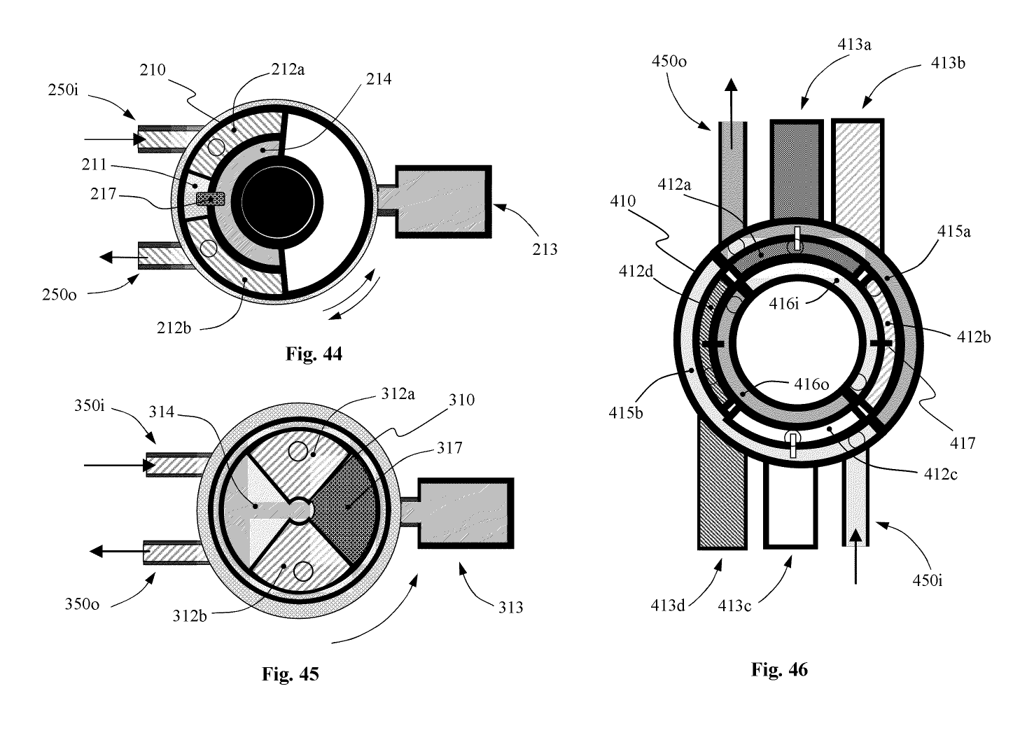

For instance, FIG. 44 shows a schematic view of a valve system having a disc (not shown) that comprises a rectilinear groove 217. The disc is rotatably mounted on a gasket 210 to be actuable by a bi-directional angular movement through an angle of 180°. The gasket 210 is fashioned as to define an inner half-ring-shaped cavity 214 connected to a piston chamber 213 and an outer half-ring-shaped part that is adjacent to said inner half-shaped cavity 214 and that is divided in two identical arcuate inlet and outlet cavities 212a, 212b by a sealing part 211. Cavities 212a, 212b are connected respectively to an inlet and an outlet port 250i, 250o of the pump. The rectilinear groove 217 is arranged on the rotatable disc such that during piston instrokes it moves along a part of gasket 210 that is adjacent to the arcuate inlet cavity 212a and the half-ring-shaped cavity 214, thereby creating a first communication allowing leakage between cavities 212a, 214 so that fluid is sucked into the piston chamber 213 during a piston instroke, while the rectilinear groove 217 moves along a part of gasket 210 that is adjacent to the arcuate outlet cavity 212b and the half-ring-shaped cavity 214, thereby creating a second communication allowing leakage between said cavities 212b, 214 so that fluid is expelled out of the piston chamber 213 during a piston outstroke.

Another example is given in FIG. 45, which shows a schematic view of a valve system comprising a disc (not shown), having an angular-sector-shaped recess 317. The disc is rotatably mounted on a gasket 310 to be actuable by a one-way angular mouvement. The gasket 310 is shaped as to define two identical angular sector-shaped cavities 312a, 312b. These cavities 312a, 312b are diametrically opposed and are connected respectively to an inlet and outlet port 350i, 350o, while two other diametrically opposed cavities 314 (one of them is hidden by the recess 317) are connected to the piston chamber 313. The recess 317 is arranged on the rotatable disc such that during piston instrokes it moves across a part of gasket 310 that is adjacent to the cavities 312a, 314 connected respectively to the inlet port 350i and the piston chamber 313, thereby creating a first communication allowing leakage between said cavities 312a, 314 so that fluid is sucked into the piston chamber 313 during a piston instroke, while during piston outstrokes the recess 317 moves across a part of gasket 310 that is adjacent to the cavities 312b, 314 connected respectively to the outlet port 350o and the piston chamber 313, thereby creating a second communication allowing leakage between said cavities 312b, 314 so that fluid is expelled out of the piston chamber 313 during a piston outstroke.

A further example is given in FIG. 46, which shows a schematic view of a valve system comprising a disc (not shown) that comprises four rectilinear grooves 417 angularly offset from each others by substantially 90 degrees. The disc is rotatably mounted on a gasket 410 that is configured to define an outer ring-shaped part divided as to form first arcuate inlet and outlet cavities 415i, 415o connected respectively to inlet and outlet ports 450i, 450o of the pump, a middle ring-shaped part divided in four arcuate chamber cavities 412a, 412b, 412c, 412d, that are each connected to a piston chamber 413a, 413b, 413c, 413d and an inner ring-shaped part divided as to form second arcuate inlet and outlet cavities 416i, 416o connected respectively to the inlet and outlet ports 450i, 450o of the pump.

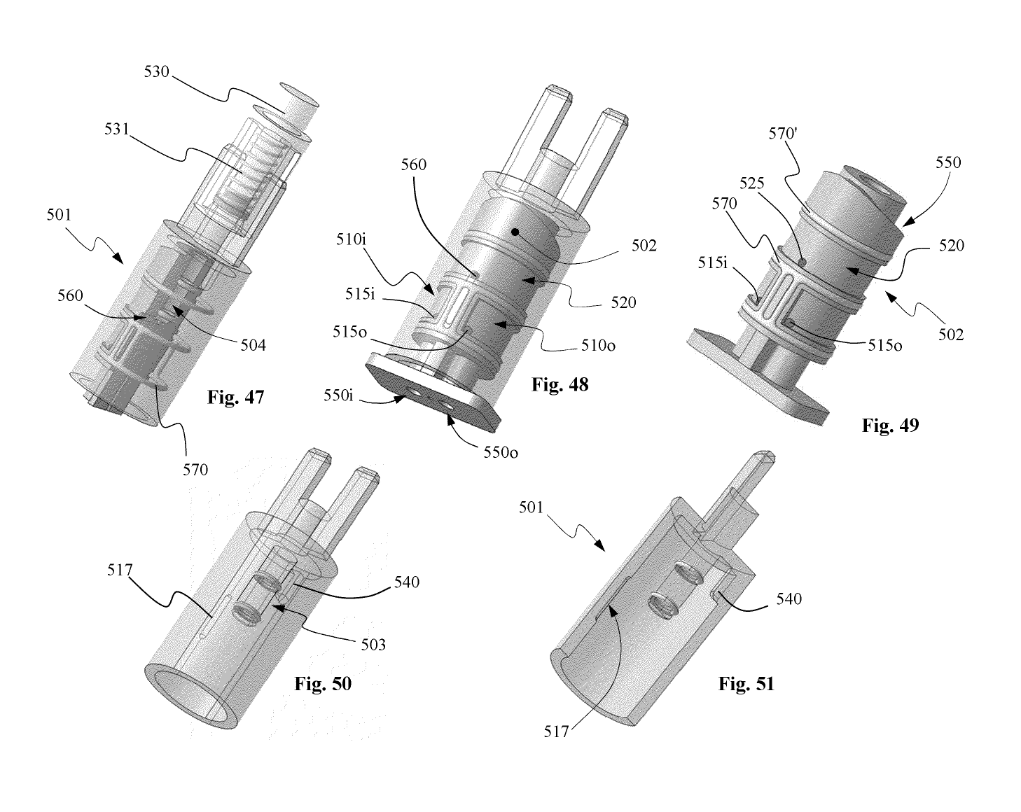

FIGS. 47 to 51 show a part of a pump having another valve configuration. This pump comprises a hollow cylindrical housing 501 that is designed to receive a cylindrical valve holder 502. A rotor 530 is adapted to impart an angular movement to the hollow cylindrical housing 501 while the cylindrical valve holder 502 remains static. A piston 503 is axially mounted within the hollow cylindrical housing 501 of the pump to project inside a corresponding cylindrical chamber 504 of the valve holder 502.

As shown particularly in FIG. 49, a first gasket 570 is arranged on the outer surface of the cylindrical valve holder 502 and is configured to define inlet and outlet cavities 510i, 510o which are opposite to each other with respect to the valve holder axis and extend preferably through 165° around said holder 502. A second gasket 570′ is arranged around the entire circumference of the cylindrical valve holder 502 so as to define an annular cavity 520 adjacent to a part of the first gasket 570 (FIG. 49).

The inlet and outlet cavities 510i, 510o are connected respectively to an inlet and an outlet port 550i, 550o of the pump by an inlet and an outlet channel 515i, 515o, while the annular cavity 520 is connected to the piston chamber 504 by a channel 560 (FIG. 47).

A rectilinear groove 517 is arranged on the inner surface of the pump housing 501 (FIG. 51) such that one part of groove 517 extends partially above the annular cavity 520, while the other part of groove 517 partially extends alternately above the inlet and outlet cavities 510i, 510o, as the pump housing 501 rotates through 360° to complete a pumping cycle.

A helical surface 550 extends around the upper part of the cylindrical valve holder 502 on an inclined plane and is designed to be in contact with a guiding projecting part 540 located inside the pump housing 501 (FIG. 51). A spring 531 is mounted at one end of the pump housing 501 (FIG. 47) whereby a to-and-fro linear movement is imparted to the latter as the guiding projecting part 540 moves along the entire circumference of the helical surface 550 when an angular movement is imparted to the pump housing 501 by the rotor 530. The spring 531 ensure that the guiding projecting part 540 is always in contact with the helical surface 550 to guarantee a right positioning of the hollow cylindrical housing 501 versus the cylindrical valve holder 502.

Different sequences of the pump of FIGS. 47 to 51 as it goes through a pumping cycle will now be described. At the beginning of a pumping cycle, the rectilinear groove 517 is arranged to move along a part of the gasket 570 that is adjacent to the inlet cavity 510i and the annular cavity 520 as the pump housing 501 rotates, thereby creating a first communication allowing leakage between said cavities 510i, 520, while the projecting part 540 of the housing 501 moves down a gradient of the helical surface 550, thereby creating a piston instroke of the pump. During said piston instroke, fluid can be sucked from the inlet port 550i, passing in turn through inlet channel 515i, inlet cavity 510i, rectilinear groove 517, annular cavity 520 and channel 560 to fill the piston chamber 504.

At the end of the piston instroke, the projecting part 540 of the pump housing 501 moves along a part of the helical surface 550 which has no gradient to ensure no movement of the piston 503 when valves switching occurs. The rectilinear groove 517 then moves along a part of the gasket 570 that is adjacent to the outlet cavity 510i and the annular cavity 520 as the pump housing 501 further rotates, thereby creating a second communication allowing leakage between cavities 510o, 520, while the projecting part 540 of housing 501 moves up a gradient of the helical surface 550, thereby creating a piston outstroke of the pump. During said piston outstroke, fluid can be released from the piston chamber 504, passing in turn through channel 560, annular cavity 520, rectilinear groove 517, outlet cavity 510o, and outlet channel 515o to be expelled out of the outlet port 550o of the pump.

It has to be noted that the rectilinear groove 517 is shaped so as to be long enough to ensure that it moves continuously above both the annular cavity 520 and the inlet and outlet cavities 510i, 510o during a pumping cycle. In a variant, one would consider adapting the pump in order to have the part adjacent to the annular chamber and the inlet and outlet cavities configured such that it follows the to-and-fro linear movement and the angular movement of the rectilinear groove 517 during a pumping cycle.

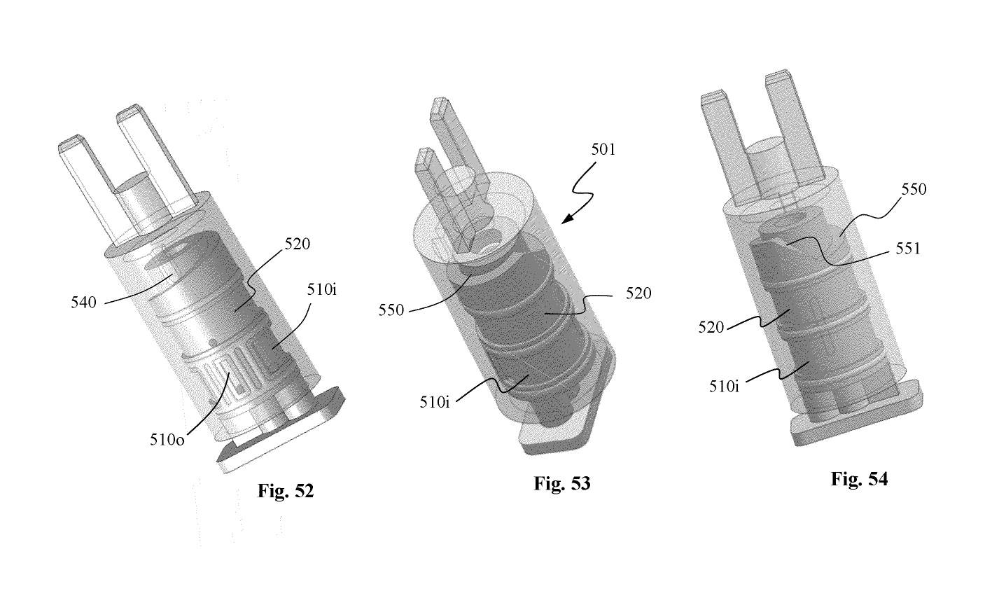

Besides, as shown by FIGS. 52 to 54, the pump can be modified to adapt the linear speed imparted to the piston 503 during its instroke to the type of fluid that needs to be pumped. In this configuration, the inlet 510i extends around the cylindrical valve holder through an angle which is preferably between 280° and 320°, while the outlet 510o extends around said valve holder through an angle which is preferably between 10° to 60°. The helical surface 550 is adapted to have a positive gradient through an angle 280° and 320° and a negative gradient 551 through an angle between 10° to 60° so that a full piston oustroke occurs when the guiding projecting part 540 moves along this negative gradient. The pump chamber can therefore be filled slowly to prevent any cavitation phenomena. This pump can be designed to be actuable clockwise and anticlockwise to be able to fill and empty the pump chamber slowly (through ˜280°) or rapidly (through ˜40°).

The size of the inlet and outlet cavities 510i, 510o as well as the profile of the helical surface can be adapted so that the filling of the piston chamber is performed by rotating the cylindrical housing 501 through an angle varying from 1 to 350 degrees.

The helical surface 550 of the cylindrical valve holder 502 or another part of the pump can be toothed so that the cylindrical housing 501 can be maintained in an axial position effortlessly by mean of a pawl in order to be suitable to be driven manually.

The pump as described in any embodiment can communicate by means of a wire or wirelessly to a remote control unit or a cellular mobile phone in order to control the amount of fluid released by the pump of fluid delivery of fluid through the micro pump and monitor internal sensors such as pressure, force, temperature, humidity or air sensor connected to the driving unit. In addition, this pump can also be connected by means of wire or wirelessly to external sensors such as a glucose sensor or any other type of sensor for providing information to the electronic in order to adapt the fluid delivery with the data sensed by the sensor. The communication protocol between the patch pump driving unit and the remote control unit can be of any type. Either the driving unit or the control unit can be programmed in order to adapt the fluid delivery accordingly to the patient inputs or sensor(s) data.

Additional elements such as vibrator or loudspeaker can be integrated to the driving unit of the pump in order to emit alarms for event such as an occlusion in the fluid line, a battery failure, a low level of drug in the reservoir or any other operational failure of the pump, including failure when any sensor detects a preset level which may present a risk to the patient.

While the invention has been described with reference to certain embodiments, it will be understood by those skilled in the art that various changes may be made and equivalents may be substituted without departing from the scope of the present invention. For instance, sealing elements can be any sort of O-ring and/or any specific gasket. Besides, any part of the pump can be machined or obtained by an injecting molding process.

Elements and/or features of different illustrative embodiments may be combined with each other and/or substituted for each other within the scope of this disclosure and appended claims. For instance, the patch pump as described in the first embodiment can be adapted to incorporate the pump according to any embodiment.

1. A micropump comprising a pump housing (1, 50, 80, 501) containing at least one piston chamber (1′, 52, 84, 504), at least one piston (2, 53, 83, 503) arranged to be linearly actuable to move back and forth inside the chamber, the micropump having at least one inlet port (13i, 60i, 86i, 550i) and at least one outlet port (15o, 60o, 86o, 550o) arranged so that a fluid can be sucked through the inlet port into the chamber during an instroke of the piston and expelled from the chamber through the outlet port during an outstroke of the piston, the pump further including a valve system, characterized in that the valve system comprises, on the one hand, at least one gasket (10, 57, 85, 570) that is shaped to define at least three cavities (12, 12a, 12b, 57i, 57o, 57a, 85a, 85i, 85o, 510i, 510o, 520) connected respectively to the piston chamber, the inlet port and the outlet port of the pump, and on the other hand, a valve switching element (16, 51, 80, 501) mounted on the gasket to allow relative movement between the gasket and the valve switching element, at least one groove (17, 67, 90, 517) or other recess (317) being arranged on the valve switching element such that, during piston instrokes, said groove or recess moves along or across a part of the gasket that is adjacent to the cavities connected respectively to the piston chamber and the inlet port of the pump, thereby creating a first communication allowing leakage between said cavities so that fluid is sucked into the piston chamber during a piston instroke, while, during piston outstrokes, said groove or recess moves along or across a part of the gasket that is adjacent to the cavities connected respectively to the piston chamber and the outlet port of the pump, thereby creating a second communication allowing leakage between said cavities so that fluid is expelled out of the piston chamber through the outlet port during a piston outstroke.

2. A micropump according to claim 1, wherein the piston chamber is a hollow elongated part, and wherein the inlet and outlet ports are arranged on the housing of the pump.

3. A micropump according to claim 1 or 2, wherein the valve switching element comprises at least one substantially rectilinear groove (17, 67, 90, 517) such that during piston instroke said groove moves along and extends across the part of the gasket (10, 57, 85, 570) that is adjacent to the cavities that are connected respectively to the piston chamber and the inlet port (13i, 60i, 86i, 550i) of the pump, while during piston outstrokes, said groove moves along and extends across the part of the gasket that is adjacent to the cavities that are connected respectively to the piston chamber and the outlet port (15o, 60o, 86o, 550o) of the pump.

4. A micropump according to claim 1 or 2, wherein the valve switching element (16, 51, 80, 501) is mounted on the gasket (10, 57, 85, 570) to allow relative rotary or to-and-fro linear movement between the gasket and the valve switching element.

5. A micropump according to claim 1 or 2, wherein the gasket (10) of the valve system comprises two concentric rings, namely an inner ring (10a) and an outer ring (10b) connected together by a first and a diametrically opposed second sealing part (11, 11′), said rings (10a, 10b) and the two sealing parts (11, 11′) defining arcuate inlet and outlet cavities (12a, 12b) connected respectively to the inlet and outlet ports (13i, 15o) of the pump, while the inner ring (10a) defines a circular cavity (12) connected to the piston chamber.

6. A micropump according to claim 1 or 2, wherein the gasket (10) of the valve system comprises two concentric rings, namely an inner ring (10a) and an outer ring (10b) connected together by a first and a diametrically opposed second sealing part (11, 11′), said rings (10a, 10b) and the two sealing parts (11, 11′) defining arcuate inlet and outlet cavities (12a, 12b) connected respectively to the inlet and outlet ports (13i, 15o) of the pump, while the inner ring (10a) defines a circular cavity (12) connected to the piston chamber, and wherein the valve switching element is a disc (16) comprising a substantially rectilinear groove (17), said disc (16) being rotatably mounted on the gasket (10) such that, during piston instrokes, said groove (17) moves along and extends radially across a part of the inner ring (10a) of the gasket (10) that is adjacent to the circular cavity (12) and the arcuate inlet cavity (12a), thereby creating a first communication allowing leakage between said cavities (12, 12a) so that fluid is sucked into the piston chamber during a piston instroke, while, during piston outstrokes, said groove (17) moves along and extends radially across a part of inner ring (10a) of the gasket (10) that is adjacent to the circular cavity (12) and the arcuate outlet cavity (12b), thereby creating a second communication allowing leakage between said cavities (12, 12b) so that fluid is expelled out of the piston chamber through the outlet port of the pump during a piston outstroke, said disc rotating through 360° during a pumping cycle.

7. A micropump according to claim 2, wherein the gasket (57) is incorporated in a substantially flat surface of the pump housing (50) and is shaped as to define inlet and outlet cavities (57i, 57o) that are connected respectively to the inlet and the outlet port (60i, 60o) of the pump, and a chamber cavity (57a) connected to the piston chamber of the pump, the inlet and outlet cavities (57i, 57o) being aligned to be adjacent to each other and to a rectilinear part of the chamber cavity (57a).

8. A micropump according to claim 7, wherein inlet and outlet cavities (57i, 57o) have substantially annular-rectangular-shaped or O-shaped borders and are adjacent to each other along their common longitudinal axis which is oriented in a direction perpendicular to the piston movement, while the chamber cavity (57a) is arranged to have its rectilinear part adjacent to one lateral side of both inlet and outlet cavities (57i, 57o).

9. A micropump according to claim 8, wherein the valve switching element (51) of the valve system has a substantially flat surface (66) and is mounted to rest on the substantially flat surface of the pump housing (50) and to allow relative to-and-fro linear movements between the valve switching element (51) and said pump housing (50), in a direction perpendicular to the piston movement, a substantially rectilinear groove (67) being arranged on the surface (66) such that, during piston instrokes, the groove (67) moves along and extends across a part of the gasket (57) that is adjacent to the O-shaped inlet cavity (57i) and the chamber cavity (57a), thereby creating a first communication allowing leakage between said cavities (57i, 57a) so that fluid is sucked into the piston chamber during the piston instroke, while, during piston outstrokes, said groove (67) moves along and extends across a part of the gasket (57) that is adjacent to the 0-shaped outlet cavity (57o) and the chamber cavity (57a), thereby creating a second communication allowing leakage between said cavities (57o, 57a) so that fluid is expelled out of the piston chamber through the outlet port of the pump during a piston outstroke.

10. A micropump according to claim 7, wherein each of the valve switching element (51) and the piston (53) comprises a guiding element (72, 72′) having a substantially rectangular aperture (71, 71′) arranged to be superposed when the valve switching element (51) is mounted on the pump housing (50), such that a part of a driving mechanism can protrude through the two apertures (71, 71′) of said guiding elements (72, 72′), said apertures (71, 71′) being arranged to have their respective longitudinal axes perpendicular to each other.

11. A micropump according to claim 2, wherein the housing (100) contains a first and a second chamber (101, 101′), and a first and a second piston (102, 102′) arranged to be linearly actuable to move back and forth inside their respective chambers (101, 101′), and wherein the gasket (110) of the valve system comprises three concentric rings (110a, 110b, 110c), namely an inner ring (110a), a middle ring (110b) and an outer ring (110c), said inner ring (110a) and middle ring (110b) being connected together by a first and a diametrically opposed second sealing part (111, 111′) as to define four cavities (112, 112a, 112b, 112c) namely a circular cavity (112) connected to the first pump chamber (101) arcuate inlet and outlet cavities (112a, 112b) symmetrically opposed and respectively connected to the inlet and outlet ports (150i, 150o) of the pump and a ring-shaped cavity (112c) connected to the second pump chamber (101′).

12. A micropump according to claim 11, wherein the valve switching element is a disc (116) comprising first and second diametrically opposed substantially rectilinear grooves (117, 117′), said disc (116) being rotatably mounted on the gasket (110) such that, during instrokes of the first piston (102) and outstrokes of the second piston (102′), the first groove (117) moves along and extends radially across a part of the inner ring (110a) of the gasket (110) that is adjacent to the circular cavity (112) and the arcuate inlet cavity (112a), thereby creating a communication allowing leakage between said cavities (112, 112a) so that fluid is sucked into the first piston chamber (101) during an instroke of the first piston (102), while the second groove (117′) moves along and extends radially across a part of the middle ring (110b) of the gasket (110) that is adjacent to the arcuate outlet cavity (112b) and the ring-shaped cavity (112c), thereby creating a communication allowing leakage between said cavities (112b, 112c) so that fluid is expelled out of the second piston chamber during an outstroke of the second piston.

13. A driving mechanism for driving a micropump according to claim 1 or 2, comprising a supporting structure (18) having a lower part adapted to receive a rotary shaft (19) around which a first rotatable element (20) is fitted, a second shaft (22) that is mounted eccentrically on rotatable element (20) and extends vertically therefrom to be connected eccentrically to a second rotatable element (23) which is axially aligned with the first rotatable element (20), the driving mechanism further comprising a sliding tray (25) whereon a piston driving pin (31) is mounted to extend vertically through the piston head of the pump, an aperture (28) being arranged on the sliding tray (25) such that the second shaft (22) protrudes vertically through said aperture (28), rotation of the rotary shaft (19) rotates eccentrically the second shaft (22), which in turn actuates a to-and-fro movement to the sliding tray (25) and the piston (2) by means of the piston driving pin (31), while the second rotatable element (23) imparts a rotating movement to the disc (16) of the valve system of the pump.

14. A driving mechanism for driving a micropump according to claim 10, comprising a rotatable element (68) mounted around the rotary shaft (69) of a motor (69′), a second shaft (70) that is eccentrically mounted on the rotatable element (68) and that is arranged to extend vertically therefrom through the two substantially rectangular apertures (71, 71′) of the superposed guiding elements (72, 72′) part of respective valve switching element (51) and piston (53) of the pump, said second shaft (70) comprising means (73, 74) to impart to-and-fro linear movements to the guiding elements (72, 72′) along the longitudinal axis of their respective substantially rectangular apertures (71, 71′) when second shaft (70) is rotating.

15. A method for manufacturing a micropump according to claim 1, by an injection moulding process which comprises the following steps: (a) injecting a mouldable plastic material capable of forming a substantially rigid element into a mould cavity assembly for obtaining a base part of the pump housing; (b) placing a seal mould matrix on said base part of the pump housing where the valve system is to be adjusted, said mould matrix being designed to reproduce the shape of the gasket of the pump; and (c) injecting into said matrix a mouldable rubber-elastic material in a flowable state, the rubber-elastic material polymerizing in the mould matrix while being bound to the pump housing to form said gasket.

16. A method for manufacturing a micropump according to claim 1, wherein a base part of the pump housing is obtained by an injection moulding process consisting of injecting a mouldable plastic material capable of forming a substantially rigid element into a mould cavity assembly for obtaining a base part of the pump housing; and wherein the gasket is obtainable by a separate injecting moulding process, and is added on a corresponding groove arranged on the base part of the pump housing.

17. In combination a micropump according to claim 1, and a driving mechanism comprising a supporting structure (18) having a lower part adapted to receive a rotary shaft (19) around which a first rotatable element (20) is fitted, a second shaft (22) that is mounted eccentrically on rotatable element (20) and extends vertically therefrom to be connected eccentrically to a second rotatable element (23) which is axially aligned with the first rotatable element (20), the driving mechanism further comprising a sliding tray (25) whereon a piston driving pin (31) is mounted to extend vertically through the piston head of the pump, an aperture (28) being arranged on the sliding tray (25) such that the second shaft (22) protrudes vertically through said aperture (28), rotation of the rotary shaft (29) rotates eccentrically the second shaft (22), which in turn actuates a to-and-fro movement to the sliding tray (25) and the piston (2) by means of the piston driving pin (31), while the second rotatable element (23) imparts a rotating movement to the disc (16) of the valve system of the pump.

18. In combination a micropump according to claim 10 and a driving mechanism comprising a rotatable element (68) mounted around the rotary shaft (69) of a motor (69′), a second shaft (70) that is eccentrically mounted on the rotatable element (68) and that is arranged to extend vertically therefrom through the two substantially rectangular apertures (71, 71′) of the superposed guiding elements (72, 72′) part of respective valve switching element (51) and piston (53) of the pump, said second shaft (70) comprising means (73, 74) to impart to-and-fro linear movements to the guiding elements (72, 72′) along the longitudinal axis of their respective substantially rectangular apertures (71, 71′) when second shaft (70) is rotating.

19. A disposable receiving unit for a patch pump comprising a case incorporating the micropump according to claim 1, and an adhesive membrane.

20. A patch pump comprising a disposable receiving unit according to claim 19 and a driving unit incorporating the driving mechanism of the pump.
